(19) United States
(12) Patent Application Publication
(10) Pub. No.: US 2026/0242362 A1
(43) Pub. Date: Aug. 20, 2026

(54) PREPARATION OF 3,4,5-TRISUBSTITUTED TRIAZOLES AND METHODS OF USING THE SAME

(71) Applicants: University of Florida Research Foundation, Incorporated, Gainesville, FL (US); The University of North Carolina at Chapel Hill, Chapel Hill, NC (US)

(72) Inventors: Ashley Elizabeth Trojniak, Carrboro, NC (US); Jeffrey Aube, Pittsboro, NC (US); Laura M. Bohn, Gainesville, FL (US)

(21) Appl. No.: 19/160,519

(22) PCT Filed: Mar. 1, 2024
(86) PCT No.: PCT/US2024/018115

§ 371 Date: Aug. 28, 2025

Related U.S. Application Data
(60) Provisional application No. 63/449,388, filed on Mar. 2, 2023.

(51) Int. Cl. C07D 405/14; A61K 31/4196; A61K 31/4439; A61K 31/4709; A61K 31/506; A61P 17/04; A61P 25/22; C07D 401/04; C07D 409/06; C07D 409/14; C07D 413/14; C07D 417/14

(52) U.S. Cl. CPC C07D 405/14; A61K 31/4196; A61K 31/4439; A61K 31/4709; A61K 31/506; A61P 17/04; A61P 25/22; C07D 401/04; C07D 409/06; C07D 409/14; C07D 413/14; C07D 417/14

(57) ABSTRACT

The present disclosure is directed to kappa opioid receptor ligands and pharmaceutical compositions thereof and their utility as neurological modulators (e.g., anti-nociceptive agents, antidepressants, anxiolytics, antipruritics). Specifically, the disclosed kappa opioid ligands are G-protein biased kappa opioid agonists containing a core and three different arms as is shown in Formula (A) below.

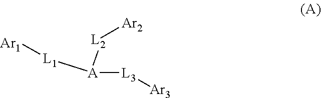

### CROSS-REFERENCE TO RELATED APPLICATIONS

This application claims the benefit of and priority under 35 U.S.C. § 119(e) to U.S. Provisional Application No. 63/449,388, filed Mar. 2, 2023, titled PREPARATION OF 3,4,5-TRISUBSTITUTED TRIAZOLES AND METHODS OF USING THE SAME, the contents of which are incorporated herewith by reference in their entirety.

### GOVERNMENT SUPPORT

This invention was made with government support under Grant No. DA031927 awarded by the National Institutes of Health. The government has certain rights in the invention.

### FIELD OF THE INVENTION

The present invention is directed to kappa opioid receptor ligands and pharmaceutical compositions thereof and their utility as neurological modulators (e.g., anti-nociceptive agents, antidepressants, anxiolytics, antipruritics).

### BACKGROUND

Opioid overdose deaths continue to be a public health issue in the United States with rates continuing to rise.¹ Opioids, including Morphine and its analogues, have bene shown to act via the μ-opioid receptor resulting in analgesia as well as physical dependence.^{2,3} One cause of the opioid crisis is due to the increased prescription of opioid pain relieving drugs that target the μ-opioid receptor resulting in addiction.

There are three opioid receptors, the μ-, δ-, and κ-opioid receptors, which are all G-protein coupled receptors. Of these receptors, the κ-opioid receptor (KOR) offers a unique opportunity to treat pain. It is distributed throughout the entire nervous system and is activated by opioid peptides, such as dynorphins. Like other opioid receptors, its activation promotes antinociception, and hence, is a target for development of pain therapeutics. KOR agonists have also been proven to be efficacious in the treatment of intractable, non-histamine-related itch, or pruritus. Currently, nalfurafine is the only clinically available KOR agonist, and it is used in the treatment of pruritus. In addition to providing pain relief without the threat of overdose, KOR agonists are unlikely to be addictive because they do not induce euphoria, nor do they promote increases in dopamine release, as abused drugs do. However, they are associated with dysphoria and sedation, which has limited their therapeutic investigation.

Thus, despite the ongoing efforts to develop analgesic pharmaceuticals with better properties compared to standard opioids, there still remains a need to discover and develop analgesic compounds that are effective and safe. Such new treatment options may involve KOR agonists devoid of negative side effects typically associated with opioids and/or side effects associated with KORs, i.e., dysphoria and sedation.

### SUMMARY

Provided herein are ligands which can bind to the κ-opioid receptor to illicit beneficial therapeutic effects (i.e., an analgesic effect) while exhibiting minimal side effects typically experienced with opioids. Thus, one aspect of the current disclosure is directed to a compound of Formula (A):

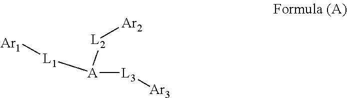

or a pharmaceutically acceptable salt thereof, wherein:
A is selected from the group consisting of

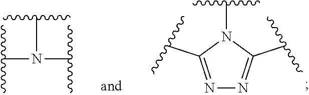

Ar₁ is selected from the group consisting of

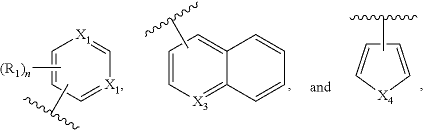

wherein X₁, X₂ and X₃ are each independently selected from the group consisting of —N— and —CH—, and X₄ is selected from the group consisting of —CH₂—, —NH—, —O—, —S—, and —N(CH₃)—, wherein R₁, in each instance, is independently selected from the group consisting of —Cl, —Br, —F, —CF₃, —OH, —CN, —NO₂, —NH₂, and (C₁-C₆)alkyl, and n is 1, 2 or 3;
Ar₂ is selected from the group consisting

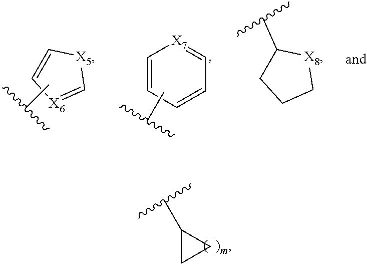

wherein X₅ and X₈ are each independently is selected from the group consisting of —CH₂—, —NH—, —O—, and —S—, and X₆ and X₇ are each independently selected from the group consisting of —N— and —CH—, and wherein m is 1, 2, 3, 4, 5, or 6;
Ar₃ is selected from the group consisting of

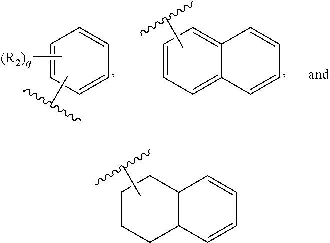

wherein R₂, in each instance, is independently selected from the group consisting of —Cl, —Br, —F, —CF₃, —OH, —CN, —NO₂, —NH₂, —O(C₁-C₆)alkyl, and —(C₁-C₆)alkyl, and q is 1, 2, 3, 4, or 5; and
L₁, L₂ and L₃ are each independently selected from the group consisting of a bond, —C(═O)—, —(CH₂)ᵣ—, —C≡C—, —CH═CH—, —C(R_{4b})(R₄ₐ)CH₂—, —CH₂C(R_{4b})(R₄ₐ)—, —C(═O)CH₂—, —CH₂CH(R₅)—, and —CH(Rₓ)—, wherein r is 1, 2, 3, 4, 5, or 6, and R₄ₐ, R_{4b}, R₅, and Rₓ are each independently selected from the group consisting of —H, —Cl, —Br, —F, —CF₃, —OH, —CN, —NO₂, —NH₂, and —(C₁-C₆)alkyl, or R₄ₐ and R_{4b} are taken together with their intervening atom(s) to form C₃-C₇ carbocyclyl.

Another aspect of the current disclosure is directed to a compound of Formula (I):

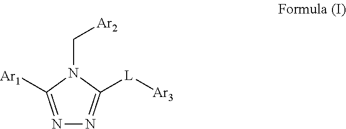

or a pharmaceutically acceptable salt thereof, wherein
Ar₁ is selected from the group consisting of

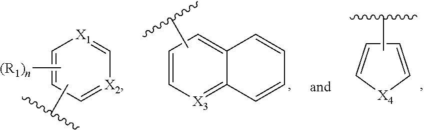

wherein X₁, X₂, and X₃ are each independently selected from the group consisting of —N— and —CH—, and X₄ is selected from the group consisting of —CH₂—, —NH—, —O—, —S—, and —N(CH₃)—, wherein Rᵢ, in each instance, is independently selected from the group consisting of, —Cl, —Br, —F, —CF₃, —OH, —CN, —NO₂, —NH₂, and —(C₁-C₆)alkyl, and n is 1, 2 or 3;
Ar₂ is selected from the group consisting of

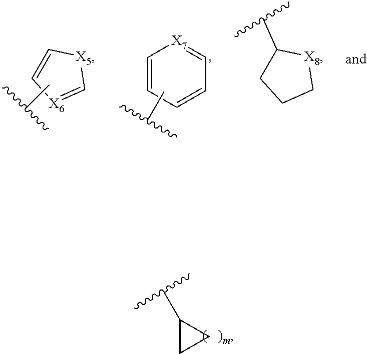

wherein X₅ and X₈ are each independently selected from the group consisting of —CH₂—, —NH—, —O—, and —S—, and X₆ and X₇ are each independently selected from the group consisting of —N— and —CH—, and wherein m is 1, 2, 3, 4, 5, or 6;
Ar₃ is selected from the group consisting of

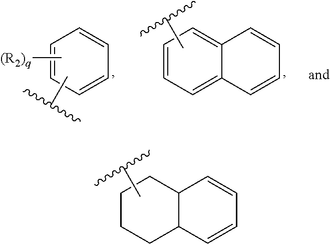

wherein R₂, in each instance, is selected from the group consisting of —Cl, —Br, —F, —CF₃, —OH, —CN, —NO₂, —NH₂, —O(C₁-C₆)alkyl, and —(C₁-C₆)alkyl, and q is 1, 2, 3, 4, or 5; and
L is selected from the group consisting of a bond, —(CH₂)ᵣ—, —C≡C—, —CH═CH—, —C(R_{4b})(R₄ₐ)CH₂—, —CH₂C(R_{4b})(R₄ₐ)—, —C(═O)CH₂—, and —CH₂CH(R₅)—, wherein r is 1, 2, 3, 4, 5, or 6, and R₄ₐ, R_{4b} and R₅ are each independently selected from the group consisting of —H, —Cl, —Br, —F, —CF₃, —OH, —CN, —NO₂, —NH₂, and —(C₁-C₆)alkyl, or R₄ₐ and R_{4b} are taken together with their intervening atom(s) to form C₃-C₇ carbocyclyl. In some embodiments, the compounds disclosed herein are κ-opioid receptor ligands. In some embodiments, the compounds disclosed herein are G-protein biased κ-opioid receptor agonists.

Another aspect of the disclosure is directed to a pharmaceutical composition comprising a compound as disclosed herein or a pharmaceutically acceptable salt thereof, and one or more pharmaceutically acceptable carrier(s).

Another aspect of the disclosure is directed to the method for treating a disease or condition mediated by the kappa opioid receptor, the method comprising administering a therapeutically effective amount of a compound disclosed herein or a pharmaceutical composition provided herein to a subject in need thereof. In some embodiments, the disease or condition to be treated is acute or chronic pain.

### DESCRIPTION OF THE DRAWINGS

FIG. 1 is a bar graph showing the national Drug-involved Overdose Death providing numbers among all ages and gender from 199-2021 (https://nida.nih.gov/research-topics/trends-statistics/overdose-death-rates).

FIG. 2 is a bar graph showing the percentage of adults aged 18 and over with chronic pain and high-impact chronic pain in the past 3 months, overall and by sex in the united states, 2019 (https://www.cdc.gov/nchs/products/databriefs/db390.htm).

FIG. 3 shows a model depicting functional selectivity of GPCR signaling. A balanced agonist would be predicted to activate multiple signaling cascades mediated by the effectors that associate with the receptor, while a biased agonist would preferentially engage with certain effectors over others to activate distinct signaling pathways.

FIGS. 4A-H show the results of testing various compounds for their ability to activate the G-protein pathway as measured through the GTPγ³⁵S binding assay vs. their ability to recruit β-arresting as measured by the galactosidase recombination assay (both described in Brust, T. B., et al. Sci. Signal 2016, 9, ra117). Compound structures are further described in more detail below.

FIG. 5 shows the results of Compound 5 tested in a standard mouse purities model. Compound 5 was administered i.p at 0.3 mg/kg and 1 mg/kg and to mice and Animals were monitored for scratching behavior post administration and compared to control animals, which were injected with vehicle.

FIG. 6 shows the results of compounds 5, U50,488H, and Triazole 1.1 tested in a standard mouse purities model. Compounds 5, U50,488H, and Triazole 1.1 were administered to mice at various doses and then monitored for scratching behavior over a one hour time frame.

FIGS. 7A-H show the results of compounds 5, U50,488H, and Triazole 1.1 tested in an open field test box mouse model for sedation. Compounds 5, U50,488H, and Triazole 1.1 were administered at various doses and animal behavior was observed, e.g., amount of distance traveled in the box over 60 minutes and amount of time spent in the center of the box over 60 minutes.

FIGS. 8A-8D show the results of compounds 5, U50,488H, and Triazole 1.1 tested in an elevated plus maze mouse for anxiety. Compounds 5, U50,488H, and Triazole 1.1 were administered at doses of 5 mg/kg, 3 mg/kg, and 15 mg/kg, respectively, and animal behavior was observed post administration, e.g., the amount of time spend in open arms, proximal open arms, distal open arms, and total distance traveled in the elevated plus maze.

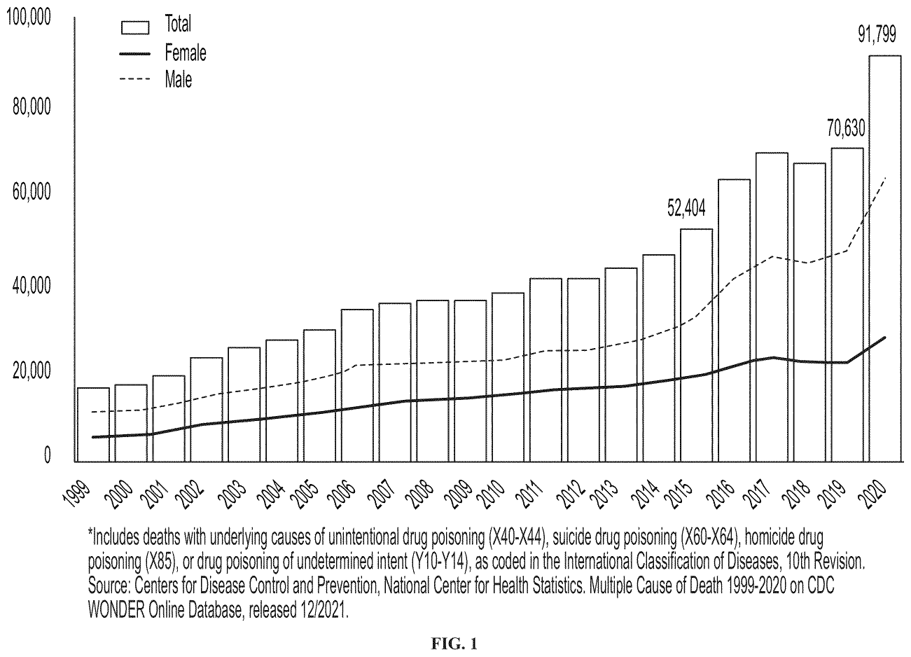

### DETAILED DESCRIPTION

The presently disclosed subject matter will now be described more fully hereinafter. However, many modifications and other embodiments of the presently disclosed subject matter set forth herein will come to mind to one skilled in the art to which the presently disclosed subject matter pertains, having the benefit of the teachings presented in the foregoing descriptions. Therefore, it is to be understood that the presently disclosed subject matter is not to be limited to the specific embodiments disclosed and that modifications and other embodiments are intended to be included within the scope of the appended claims. In other words, the subject matter described herein covers all alternatives, modifications, and equivalents. In the event that one or more of the incorporated literature, patents, and similar materials differs from or contradicts this application, including but not limited to defined terms, term usage, described techniques, or the like, this application controls. Unless otherwise defined, all technical and scientific terms used herein have the same meaning as commonly understood by one of ordinary skill in this field. All publications, patent applications, patents, and other references mentioned herein are incorporated by reference in their entirety.

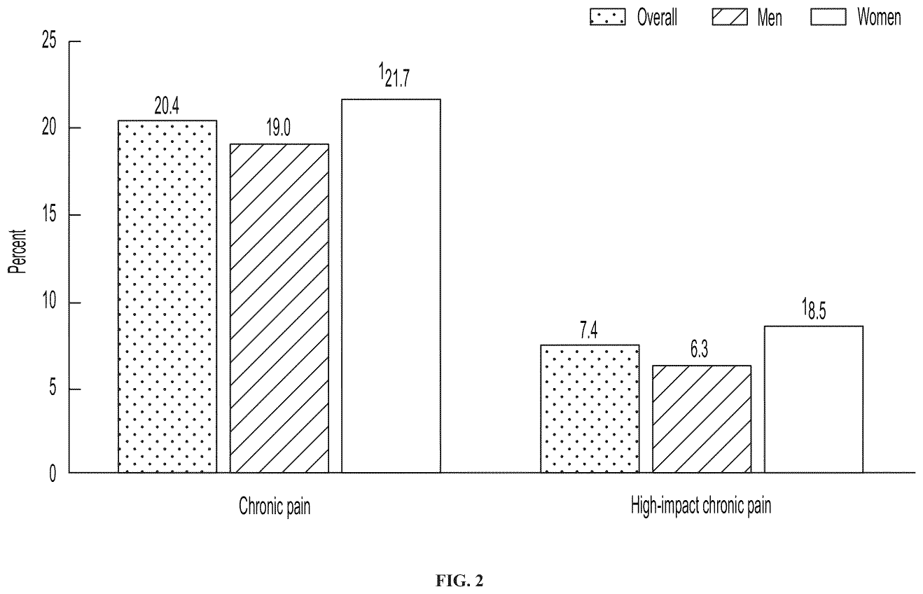

Opioid addiction presents a major public health crisis in the United States. Often opioid addiction occurs when opioids are initially prescribed for the treatment of acute and chronic pain in patients. For example, in 2019, 20.4% of adults had chronic pain and 7.4% of adults had chronic pain that frequently limited life or work activities (referred to as high impact chronic pain) in the past 3 months. The percentage of adults aged 18 and over with chronic pain and high impact chronic pain in the past 3 month are shown in FIG. 2.

Kappa opioid receptor (KOR) agonists represent promising therapeutics for pain relief due to their strong analgesic properties along with their non-addictive side-effect profile. In addition, KOR is responsible for regulating a wide variety of physiological functions other than pain relief,⁴ such as itch relief,^{5, 6} addiction,⁷ depression,⁸ and immune response.⁹ Unfortunately, KOR agonists have also been associated with dysphoria and sedation, which has limited their therapeutic investigation.

However, despite these liabilities associated with the KOR, numerous small molecule KOR agonists have been reported including U-69,593,¹⁴ ketazocine,¹⁵ 6′-guanidinonaltrindole,¹⁶ and salvinorin A,¹⁷ which was the first small molecule KOR agonist that did not contain a basic nitrogen. These small molecule KOR agonists are shown below:

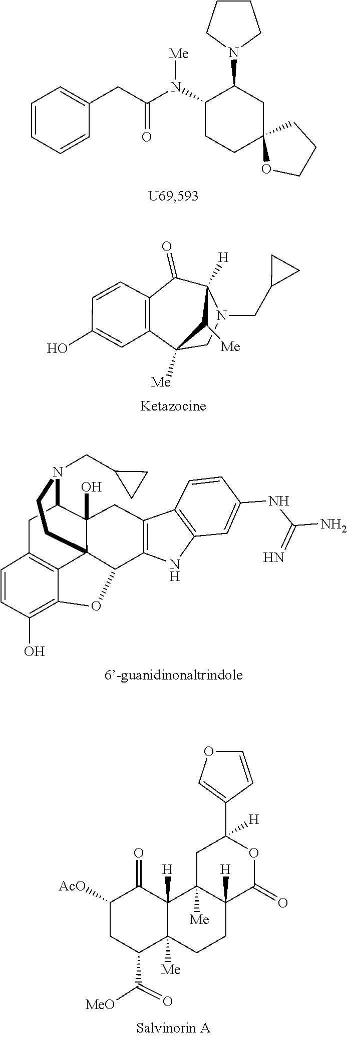

In addition, three crystal structures have been reported for the KOR, one with the JDTic antagonist¹⁸ and one as a nano-body active state conformer.¹⁹ Recently, a structure of the KOR bound to its native dynorphin peptide ligand was published.²⁰

Furthermore, four KOR agonist chemotypes were identified via High Throughput Screening, including a triazole chemotype.²¹ In particular, Triazole 1.1 was identified as a potent, G-protein biased KOR agonist.²² In vivo studies in mice show evidence that in comparison with balanced KOR agonist U50,488H, Triazole 1.1 has similar analgesic and anti-itch properties while avoiding sedation effects traditionally associated with the KOR.²³ Similar results have been found in non-human primates.^{24,25} In addition, Triazole 1.1 has been found to reduce oxycodone self-administration in male rats.²⁶ These studies show that G-protein biased KOR agonists are able to untangle the desired analgesic properties from the undesired side-effects such as sedation and dysphoria which are typically associated with the KOR.

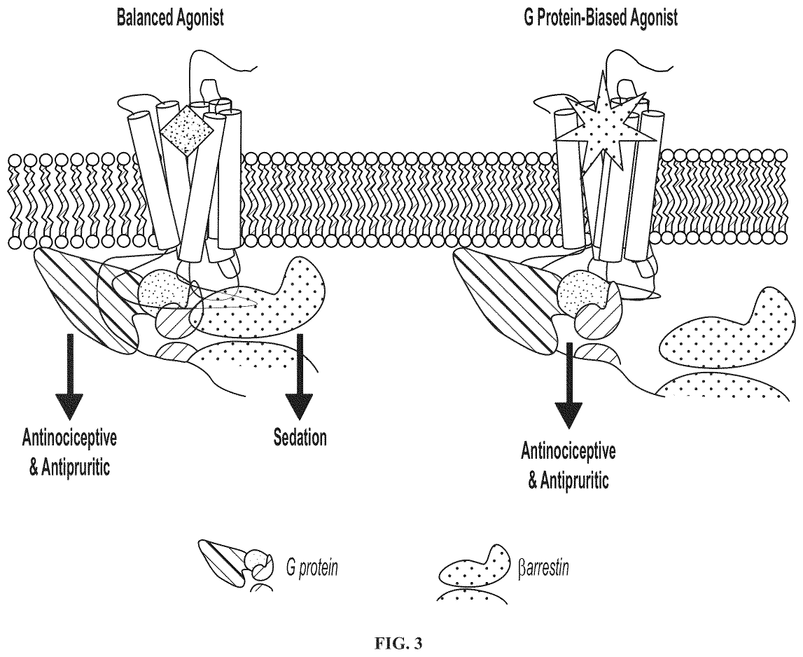

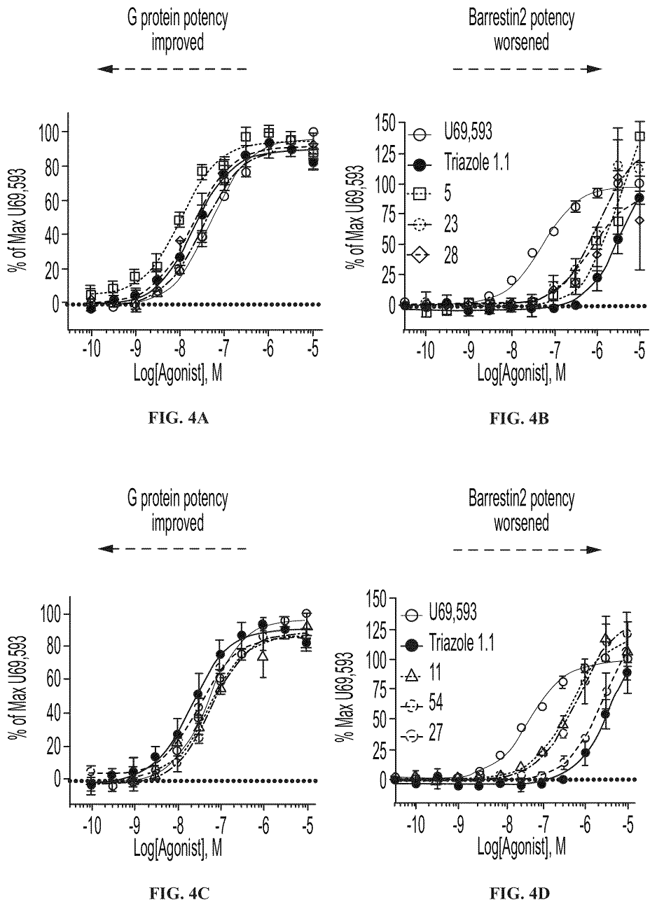

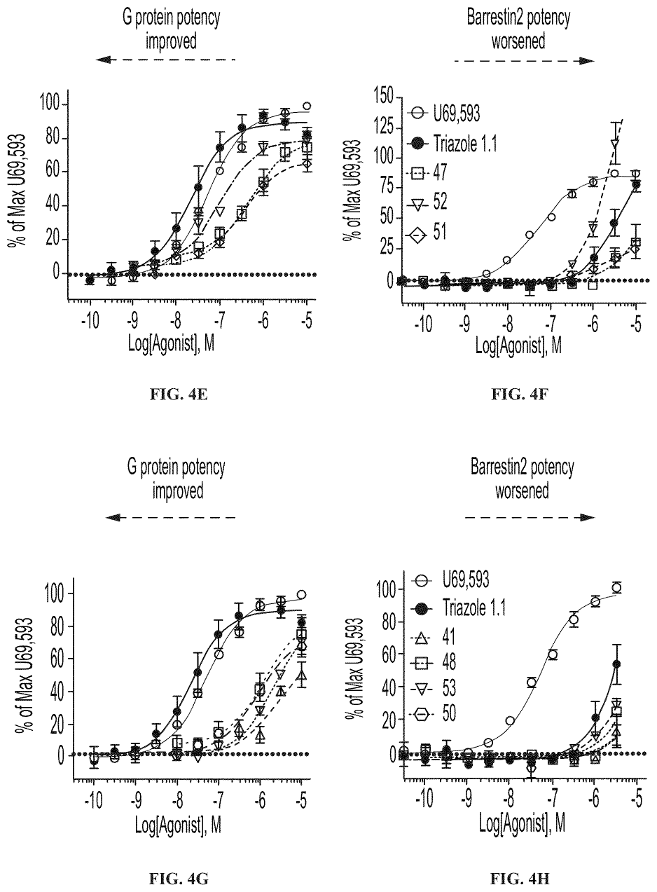

Here such “untangling” of the efficacious behavior of the KOR agonist from its undesired side effects is the result of G protein activation and β-arresting function at the same time. The concept of being able to separate these two activities and/or pathways is generally known as “functional selectivity” or “ligand bias” and compounds can exhibit a range of activities from fully “balanced” (i.e., that activate each pathway with comparable efficacy) to highly “biased” displaying preference for engaging one signaling pathway over another. Here, a suitable compound would be able to function as a nociceptive agent and/or antipruritic agent without the dysphoria, sedation and other side effects typically associated with the GPCR target, i.e., the kappa opioid receptor (FIG. 3).

Over the years, Triazole 1.1 has undergone various Structure-Activity Relationship Studies, mainly focusing on the three arms of the triazole.^{27,28} Although Triazole 1.1 is potent, biased, and active in vivo, the pharmacokinetic profile needs to be optimized.²²

For instance, the sulfur-containing side-chain in the scaffold of Triazole 1.1 was sought of being one of the possible metabolically liable site as the as the sulfur can be oxidized to the sulfoxide or the sulfone. As such, triazole analogues of Triazole 1.1 in which the sulfur side-chain was replaced with a carbon side-chain to give 3,4,5 trisubstituted 1,2,4 triazoles were prepared as is shown in the scheme below:

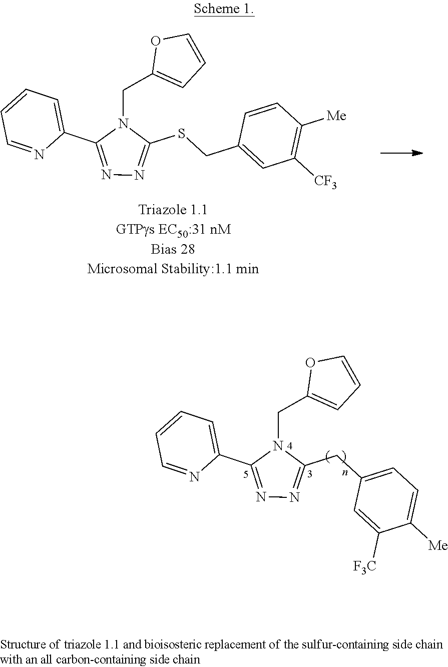

However, metabolism studies revealed that removing the heteroatom from the side chain did not have a significant effect on improving the microsomal stability of Triazole 1.1.

Furthermore, these metabolic studies indicated that the sulfur atom is not the main site of metabolic instability. What was surprising and unexpected, however, is that the triazole analogues of triazole 1.1 exhibited similar KOR binding affinity as Triazole 1.1, indicating the sulfur atom is not electronically modulating the triazole ring. Thus, the KOR ligands, as disclosed herein, represent a viable bioisosteric replacement for Triazole 1.1.

Pharmaceutical compositions containing KOR ligands and methods of use thereof are described further in more detail below.

### A. Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Michael B. Smith, March's Advanced Organic Chemistry, 7^{th} Edition, John Wiley & Sons, Inc., New York, 2013; Richard C. Larock, Comprehensive Organic Transformations, John Wiley & Sons, Inc., New York, 2018; and Carruthers, Some Modem Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, IN 1972). The present disclosure additionally encompasses compounds as individual isomers substantially free of other isomers, and alternatively, as mixtures of various isomers.

Unless otherwise provided, formulae and structures depicted herein include compounds that do not include isotopically enriched atoms, and also include compounds that include isotopically enriched atoms. For example, compounds having the present structures except for the replacement of hydrogen by deuterium or tritium, replacement of ¹⁹F with ¹⁸F, or the replacement of a carbon by a ¹³C- or ¹⁴C-enriched carbon are within the scope of the disclosure. Such compounds are useful, for example, as analytical tools or probes in biological assays.

When a range of values (“range”) is listed, it encompasses each value and sub-range within the range. A range is inclusive of the values at the two ends of the range unless otherwise provided. For example, “C₁₋₆ alkyl” encompasses, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

As used herein, the term “alkyl group” or “alkyl” refers to a saturated hydrocarbon radical containing 1 to 8, 1 to 6, 1 to 4, or 5 to 8 carbons. In some embodiments, the saturated radical contains more than 8 carbons. An alkyl group is structurally similar to a noncyclic alkane compound modified by the removal of one hydrogen from the noncyclic alkane and the substitution therefore of a non-hydrogen group or radical. Alkyl group radicals can be branched or unbranched. Lower alkyl group radicals have 1 to 4 carbon atoms. Higher alkyl group radicals have 5 to 8 carbon atoms. Examples of alkyl, lower alkyl, and higher alkyl group radicals include, but are not limited to, methyl, ethyl, n-propyl, isopropyl, n-butyl, sec butyl, t butyl, amyl, t amyl, n-pentyl, n-hexyl, i-octyl and like radicals.

The term “alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, isobutyl), pentyl (C₅) (e.g., n-pentyl, 3-pentanyl, amyl, neopentyl, 3-methyl-2-butanyl, tert-amyl), and hexyl (C₆) (e.g., n-hexyl). Additional examples of alkyl groups include n-heptyl (C₇), n-octyl (C₈), n-dodecyl (C₁₂), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₂ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu or s-Bu), unsubstituted isobutyl (i-Bu)). In certain embodiments, the alkyl group is a substituted C₁₋₁₂ alkyl (such as substituted C₁₋₆ alkyl, e.g., —CH₂F, —CHF₂, —CF₃, —CH₂CH₂F, —CH₂CHF₂, —CH₂CF₃, or benzyl (Bn)).

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 14 ring carbon atoms (“C₃₋₁₄ carbocyclyl”). In some embodiments, a carbocyclyl group has 3 to 13 ring carbon atoms (“C₃₋₁₃ carbocyclyl”). In some embodiments, a carbocyclyl group has 3 to 12 ring carbon atoms (“C₃₋₁₂ carbocyclyl”). In some embodiments, a carbocyclyl group has 3 to 11 ring carbon atoms (“C₃₋₁₁ carbocyclyl”). In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C_{3.10} carbocyclyl groups include the aforementioned C_{3.8} carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. Exemplary C₃₋₈ carbocyclyl groups include the aforementioned C_{3.10} carbocyclyl groups as well as cycloundecyl (C₁₁), spiro[5.5]undecanyl (C₁₁), cyclododecyl (C₁₂), cyclododecenyl (C₁₂), cyclotridecane (C₁₃), cyclotetradecane (C₁₄), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and, in some embodiments, are saturated or contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl. In certain embodiments, the carbocyclyl includes 0, 1, or 2 C═C double bonds in the carbocyclic ring system, as valency permits.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which is substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any of the substituents described herein that results in the formation of a stable compound. The present disclosure contemplates any and all such combinations in order to arrive at a stable compound. For purposes of this disclosure, heteroatoms such as nitrogen may have hydrogen substituents and/or any suitable substituent as described herein which satisfy the valencies of the heteroatoms and results in the formation of a stable moiety. The disclosure is not limited in any manner by the exemplary substituents described herein.

As used herein, the designations “C(═O),” “CO” and “C(O)” are used to indicate a carbonyl moiety. Examples of suitable carbonyl moieties include, but are not limited to, those found in ketones and aldehydes.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease.

As used herein, the terms “co-administration” and “co-administering” refer to the administration of at least two agent(s) (e.g., a KOR agonist as disclosed herein and one or more additional therapeutics) or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent.

As used herein, the term “pharmaceutical composition” refers to the combination of an active agent with a carrier, inert or active, making the composition especially suitable for diagnostic or therapeutic use in vitro, in vivo or ex vivo.

The terms “pharmaceutically acceptable” or “pharmacologically acceptable,” as used herein, refer to compositions that do not substantially produce adverse reactions, e.g., toxic, allergic, or immunological reactions, when administered to a subject.

As used herein, the term “pharmaceutically acceptable carrier” refers to any of the standard pharmaceutical carriers including, but not limited to, phosphate buffered saline solution, water, emulsions (e.g., such as an oil/water or water/oil emulsions), and various types of wetting agents, any and all solvents, dispersion media, coatings, sodium lauryl sulfate, isotonic and absorption delaying agents, disintigrants (e.g., potato starch or sodium starch glycolate), and the like. The compositions also can include stabilizers and preservatives. For examples of carriers, stabilizers and adjuvants, see, e.g., Martin, Remington's Pharmaceutical Sciences, 15th Ed., Mack Publ. Co., Easton, Pa. (1975), incorporated herein by reference in its entirety.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of the present disclosure include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence. As used herein, a subject is “in need of” a treatment if such subject would benefit biologically, medically or in quality of life from such treatment.

An “effective amount” of a compound described herein refers to an amount sufficient to elicit the desired biological response. An effective amount of a compound described herein may vary depending on such factors as the desired biological endpoint, severity of side effects, disease, or disorder, the identity, pharmacokinetics, and pharmacodynamics of the particular compound, the condition being treated, the mode, route, and desired or required frequency of administration, the species, age and health or general condition of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses. In certain embodiments, the desired dosage is delivered three times a day, two times a day, once a day, every other day, every third day, every week, every two weeks, every three weeks, or every four weeks. In certain embodiments, the desired dosage is delivered using multiple administrations (e.g., two, three, four, five, six, seven, eight, nine, ten, eleven, twelve, thirteen, fourteen, or more administrations).

In certain embodiments, an effective amount of a compound for administration one or more times a day to a 70 kg adult human comprises about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

In certain embodiments, the compounds of the present disclosure are administered orally or parenterally at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic effect.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent.

The terms “condition,” “disease,” and “disorder” are used interchangeably.

### B. Compounds

Provided herein are kappa opioid receptor ligands and methods of use thereof for the treatment of a disease or condition associated with kappa opioid receptors, such as acute and chronic pain, pruritis, addiction and mood disorders. In some embodiments, the KOR ligands are agonists. In some embodiments, the KOR ligands are G-protein biased KOR agonists.

In one aspect, provided herein is a compound of Formula (A):

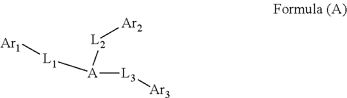

or a pharmaceutically acceptable salt thereof, wherein
   A is selected from the group consisting of

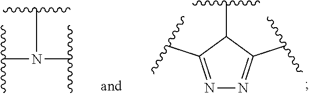

Ar₁ is selected from the group consisting of

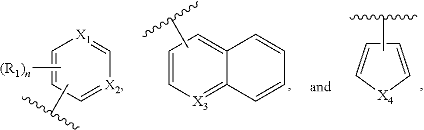

wherein X₁, X₂ and X₃ are each independently selected from the group consisting of —N— and —CH—, and X₄ is selected from the group consisting of —CH₂—, —NH—, —O—, —S—, and —N(CH₃)—, wherein R₁, in each instance, is independently selected from the group consisting of —Cl, —Br, —F, —CF₃, —OH, —CN, —NO₂, —NH₂, and (C₁-C₆)alkyl, and n is 1, 2 or 3;
Ar₂ is selected from the group consisting of

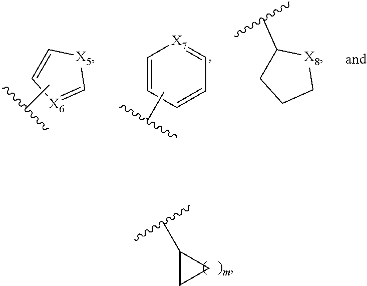

wherein X₅ and X₈ are each independently is selected from the group consisting of —CH₂—, —NH—, —O—, and —S—, and X₆ and X₇ are each independently selected from the group consisting of —N— and —CH—, and wherein m is 1, 2, 3, 4, 5, or 6;
Ar₃ is selected from the group consisting of

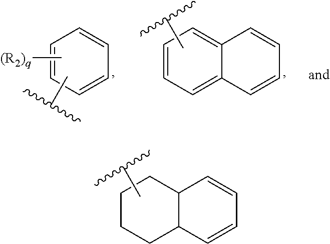

wherein R₂, in each instance, is independently selected from the group consisting of —Cl, —Br, —F, —CF₃, —OH, —CN, —NO₂, —NH₂, —O(C₁-C₆)alkyl, and —(C₁-C₆)alkyl, and q is 1, 2, 3, 4, or 5; and
L₁, L₂ and L₃ are each independently selected from the group consisting of a bond, —C(═O)—, —(CH₂)ᵣ—, —C≡C—, —CH═CH—, —C(R_{4b})(R₄ₐ)CH₂—, —CH₂C(R_{4b})(R₄ₐ)—, —C(═O)CH₂—, —CH₂CH(R₅)—, and —CH(Rₓ)—, wherein r is 1, 2, 3, 4, 5, or 6, and R₄ₐ, R_{4b}, R₅, and Rₓ are each independently selected from the group consisting of —H, —Cl, —Br, —F, —CF₃, —OH, —CN, —NO₂, —NH₂, and —(C₁-C₆)alkyl, or R₄ₐ and R_{4b} are taken together with their intervening atom(s) to form C₃-C₇ carbocyclyl.

In some embodiments, the compound of Formula (I) has the structure of Formula (A-1):

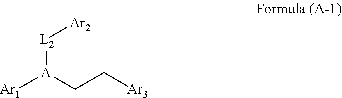

or a pharmaceutically acceptable salt thereof, wherein L₂ is a bond, —(CH₂)ᵣ—, or —CH(R^{x})—, wherein r is 1 or 2, and R^{x} is selected from the group consisting of —H and —(C₁-C₆)alkyl.

In one aspect, provided herein is a compound with the structure of Formula (I):

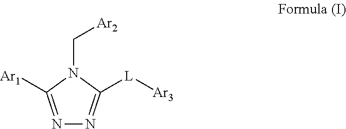

or a pharmaceutically acceptable salt thereof, wherein
Ar₁ is selected from the group consisting of

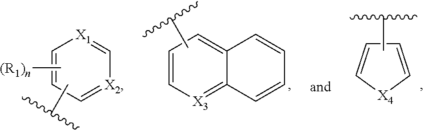

wherein X₁, X₂, and X₃ are each independently selected from the group consisting of —N— and —CH—, and X₄ is selected from the group consisting of —CH₂—, —NH—, —O—, —S—, and —N(CH₃)—, wherein R₁, in each instance, is independently selected from —Cl, —Br, —F, —CF₃, —OH, —CN, —NO₂, —NH₂, and —(C₁-C₆)alkyl, and n is 1, 2, or 3;
Ar₂ is selected from the group consisting of

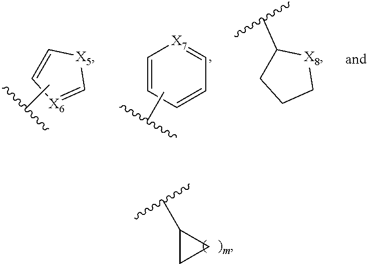

wherein X₅ and X₈ are each independently selected from the group consisting of —CH₂—, —NH—, —O—, and —S—, and X₆ and X₇ are each independently selected from the group consisting of —N— and —CH—, and wherein m is 1, 2, 3, 4, 5, or 6;
Ar₃ is selected from the group consisting of

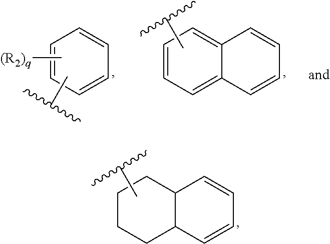

wherein R₂, in each instance, is independently selected from —Cl, —Br, —F, —CF₃, —OH, —CN, —NO₂, —NH₂, —O(C₁-C₆)alkyl, and —(C₁-C₆)alkyl, and q is 1, 2, 3, 4, or 5; and
L is selected from the group consisting of a bond, —(CH₂)ᵣ—, —C≡C—, —CH═CH—, —C(R_{4b})(R₄ₐ)CH₂—, —CH₂C(R_{4b})(R₄ₐ)—, —C(═O)CH₂—, and —CH₂CH(R₅)—, wherein r is 1, 2, 3, 4, 5, or 6, and R₄ₐ, R_{4b}, and R₅ are each independently selected from —H, —Cl, —Br, —F, —CF₃, —OH, —CN, —NO₂, —NH₂, and —(C₁-C₆)alkyl, or R₄ₐ and R_{4b} are taken together with their intervening atom(s) to form C₃-C₇ carbocyclyl. In some embodiments, the compounds disclosed herein are κ-opioid receptor ligands. In some embodiments, the compounds disclosed herein are G-protein biased κ-opioid receptor agonists.

In some embodiments, L is a bond.

In some embodiments, L is —(CH₂)ᵣ—, wherein r is 1, 2 or 3. In some embodiments, In some embodiments, L is —(CH₂)ᵣ and r is 2.

In some embodiments, L is —C≡C—.

In some embodiments, L is an olefin such as —CH═CH—. In some embodiments, the olefin is a trans-olefin. In some embodiments, the olefin is a cis-olefin. In some embodiments, L is —CH═CH—.

In some embodiments, L is selected from —C(R_{4b})(R₄ₐ)CH₂—, —CH₂C(R_{4b})(R₄ₐ)—, —C(═O)CH₂—, and —CH₂CH(R₅)—, wherein R₄ₐ, R_{4b}, and R₅ are each independently selected from —H, —F, and —OH, or R₄ₐ and R_{4b} are taken together with their intervening atom(s) to form C₃-C₇ carbocyclyl. In some embodiments, L is selected from —C(R_{4b})(R₄ₐ)CH₂—, —CH₂C(R_{4b})(R₄ₐ)—, —C(═O)CH₂—, and —CH₂CH(R₅)—, wherein R₄ₐ, R_{4b}, and R₅ are each independently selected from —H, —F, and —OH.

In some embodiments, Ar₁ is

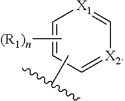

In some embodiments, X₂ is —CH—. In some embodiments, X₂ is —CH— and X₁ is —N. In some embodiments, X₁ and X₂ are each —CH—. In some embodiments, R₁, in each instance, is —CH₃ and n is 1.

In some embodiments, Ar₁ is

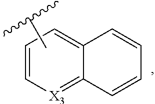

wherein X₃ is —N— or —CH—. In some embodiments, X₃ is —N—. In some embodiments, X₃ is —CH—.

In some embodiments, Ar₁ is

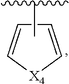

wherein X₄ is selected from —CH₂—, —NH—, —O—, —S—, and —N(CH₃)—. In some embodiments, X₄ is —CH₂—. In some embodiments, X₄ is —NH—. In some embodiments, X₄ is —O—. In some embodiments, X₄ is —S—. In some embodiments, X₄ is —N(CH₃)—.

In some embodiments, Ar₁ is selected from the group consisting of:

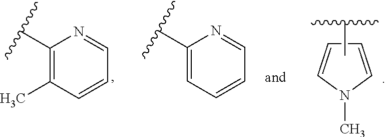

In some embodiments, Ar₁ is

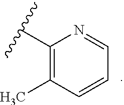 [SMILES: *c1ncccc1C]

In some embodiments, Ar₁ is

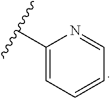 [SMILES: *c1ccccn1]

In some embodiments, Ar₁ is

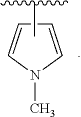

In some embodiments, the compound of Formula (I) has the structure of Formula (II):

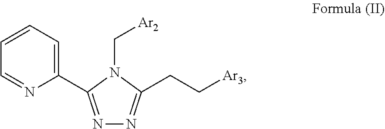

or a pharmaceutically acceptable salt thereof.

In some embodiments, Ar₃ is

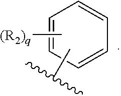

In some embodiments, R₂, in each instance, is independently selected from —Cl, —Br, —CF₃, and —(C₁-C₆)alkyl. In some embodiments, R₂, in each instance, is independently selected from —Cl, and —Br. In some embodiments, R₂, in each instance is independently selected from —CF₃ and —(C₁-C₆)alkyl. In some embodiments, q is 1 or 2. In some embodiments, Ar₃ is selected from the group consisting of

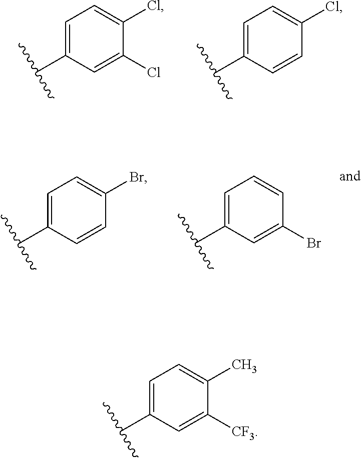 [SMILES: *c1ccc(Cl)c(Cl)c1 ; *c1ccc(Cl)cc1 ; *c1ccc(Br)cc1 ; *c1cccc(Br)c1 ; *c1ccc(C)c(C(F)(F)F)c1]

In some embodiments, Ar₃ is

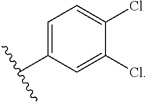 [SMILES: *c1ccc(Cl)c(Cl)c1]

In some embodiments, Ar₃ is

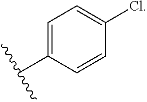 [SMILES: *c1ccc(Cl)cc1]

In some embodiments, Ar₃ is

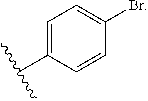 [SMILES: *c1ccc(Br)cc1]

In some embodiments, Ar₃ is

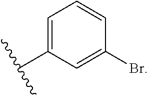 [SMILES: *c1cccc(Br)c1]

In some embodiments, Ar₃ is

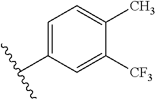 [SMILES: *c1ccc(C)c(C(F)(F)F)c1]

In some embodiments, the compound of Formula (II) has the structure of Formula (III):

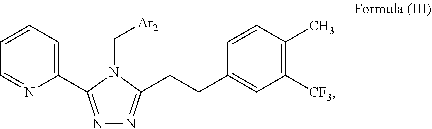

or a pharmaceutically acceptable salt thereof.

In some embodiments, Ar₂ is

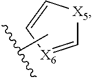

X₅ is selected from —NH—, —O—, and —S—.

In some embodiments, Ar₂ is

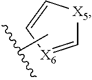

X₅ is selected from —NH—, —O—, and —S— and X₆ is —CH—.

In some embodiments, Ar₂ is

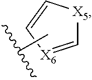

X₅ is selected from —NH—, —O—, and —S— and X₆ is —N—. In certain embodiments, Ar₂ is

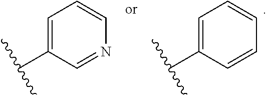 [SMILES: *c1cccnc1 ; *c1ccccc1]

In some embodiments, Ar₂ is selected from the group consisting of

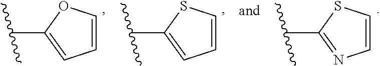

In some embodiments, Ar₂ is

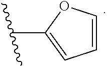 [SMILES: *c1ccco1]

In some embodiments, Ar₂ is

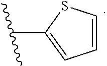 [SMILES: *c1cccs1]

In some embodiments, Ar₂ is

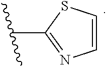 [SMILES: *c1nccs1]

In some embodiments, Ar₂ is

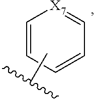

wherein X₇ is —N— or —CH—. In some embodiments, Ar₂ is

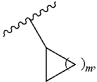

wherein m is 1, 2, or 3.

In some embodiments, the compound of Formula (A) or Formula (I) has the structure:

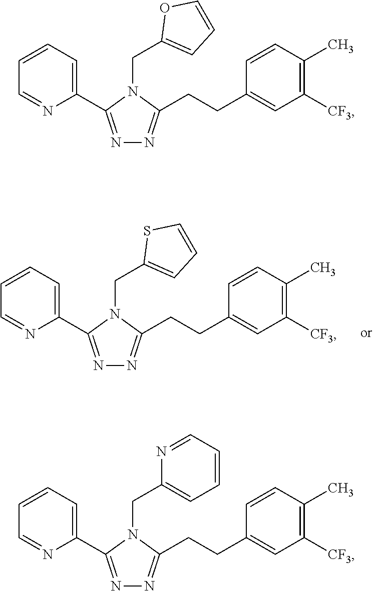 [SMILES: Cc1ccc(CCc2nnc(-c3ccccn3)n2Cc2ccco2)cc1C(F)(F)F ; Cc1ccc(CCc2nnc(-c3ccccn3)n2Cc2cccs2)cc1C(F)(F)F ; "or" ; Cc1ccc(CCc2nnc(-c3ccccn3)n2Cc2ccccn2)cc1C(F)(F)F]

or a pharmaceutically acceptable salt thereof.

In certain embodiments, the compound of Formula (A) has the structure:

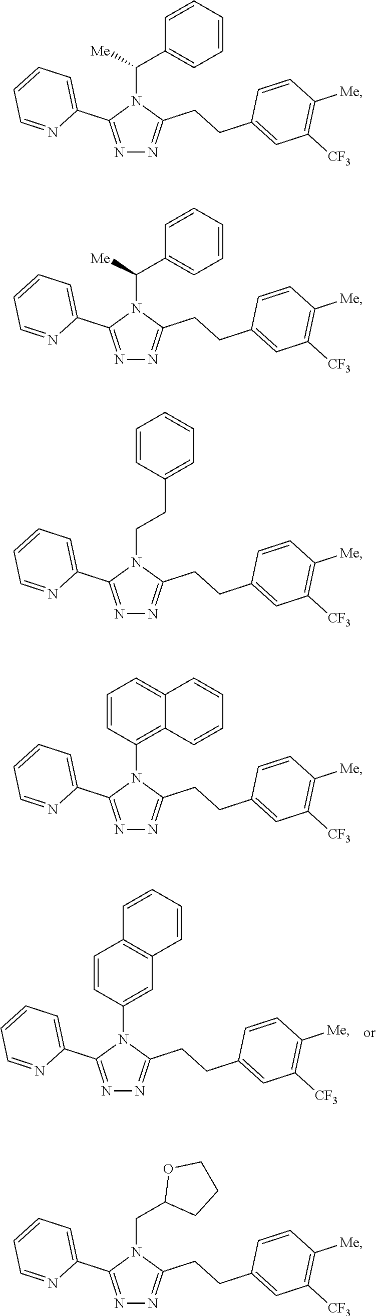 [SMILES: Cc1ccc(CCc2nnc(-c3ccccn3)n2[C@H](C)c2ccccc2)cc1C(F)(F)F ; Cc1ccc(CCc2nnc(-c3ccccn3)n2[C@@H](C)c2ccccc2)cc1C(F)(F)F ; Cc1ccc(CCc2nnc(-c3ccccn3)n2CCc2ccccc2)cc1C(F)(F)F ; Cc1ccc(CCc2nnc(-c3ccccn3)n2-c2cccc3ccccc23)cc1C(F)(F)F ; Cc1ccc(CCc2nnc(-c3ccccn3)n2-c2ccc3ccccc3c2)cc1C(F)(F)F ; Cc1ccc(CCc2nnc(-c3ccccn3)n2CC2CCCO2)cc1C(F)(F)F]

or a pharmaceutically acceptable salt thereof.

The compounds described herein may, in some cases, exist as diastereomers, enantiomers, or other stereoisomeric forms. The compounds presented herein include all diastereomeric, enantiomeric, and epimeric forms as well as the appropriate mixtures thereof. Separation of stereoisomers may be performed by chromatography and/or recrystallization or by the forming diastereomers and separation thereof (Jean Jacques, Andre Collet, Samuel H. Wilen, “Enantiomers, Racemates and Resolutions”, John Wiley And Sons, Inc., 1981). Stereoisomers may also be obtained by stereoselective synthesis using synthetic methods known in the art. In some embodiments, the compounds disclosed herein are enantiomers having an enantiomeric excess (% ee) of at least about 50%, about 60%, about 70%, about 80%, about 90%, about 95%, about 98%, or about 99.5%. In some embodiments, the compounds disclosed herein are diastereomers having a diastereomeric excess (% de) of at least about 50%, about 60%, about 70%, about 80%, about 90%, about 95%, about 98%, or about 99.5%. In some embodiments, the compounds disclosed herein are present as enantiomeric or diastereomeric mixtures.

The methods and compositions described herein include the use of amorphous forms as well as crystalline forms (also known as polymorphs). The compounds described herein may be in the form of pharmaceutically acceptable salts. Active metabolites of these compounds having the same type of activity are included in the scope of the present disclosure.

In some embodiments, the compounds described herein may be formed as, and/or used as, pharmaceutically acceptable salts. The type of pharmaceutical acceptable salts, include, but are not limited to: (1) acid addition salts, formed by reacting the free base form of the compound with a pharmaceutically acceptable: inorganic acid, such as, for example, hydrochloric acid, hydrobromic acid, sulfuric acid, phosphoric acid, metaphosphoric acid, and the like; or with an organic acid, such as, for example, acetic acid, propionic acid, hexanoic acid, cyclopentanepropionic acid, glycolic acid, pyruvic acid, lactic acid, malonic acid, succinic acid, malic acid, maleic acid, fumaric acid, trifluoroacetic acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, toluenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, butyric acid, phenylacetic acid, phenylbutyric acid, valproic acid, and the like; (2) salts formed when an acidic proton present in the parent compound is replaced by a metal ion, e.g., an alkali metal ion (e.g. lithium, sodium, potassium), an alkaline earth ion (e.g. magnesium, or calcium), or an aluminum ion. In some cases, compounds described herein may coordinate with an organic base, such as, but not limited to, ethanolamine, diethanolamine, triethanolamine, tromethamine, N-methylglucamine, dicyclohexylamine, tris(hydroxymethyl)methylamine. In other cases, compounds described herein may form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, sodium hydroxide, and the like.

In some embodiments, the compounds and salts described herein include isotopically labeled compounds. In general, isotopically labeled compounds are identical to those recited in the various formulae and structures presented herein, but for the fact that one or more atoms are replaced by an atom having an atomic mass or mass number different from the atomic mass or mass number most common in nature. Examples of isotopes that can be incorporated into the present compounds include isotopes of hydrogen, carbon, nitrogen, oxygen, fluorine and chlorine, for example, ²H, ³H, ¹³C, ¹⁴C, ¹⁵N, ¹⁸O, ¹⁷O, ³⁵S, ¹⁸F, ³⁶Cl, respectively. Certain isotopically labeled compounds described herein, for example, those into which radioactive isotopes such as ³H and ¹⁴C are incorporated, are useful in drug and/or substrate tissue distribution assays. Further, substitution with isotopes such as deuterium, i.e., ²H, can afford certain therapeutic advantages resulting from greater metabolic stability, such as, for example, increased in vivo half-life or reduced dosage requirements.

In some embodiments, the compounds disclosed herein are selective KOR binding ligands. As used herein, the term “selective binding ligands” refers to compounds, which display an increased selectivity for binding a particular KOR when compared to inhibiting other opioid receptors (such as μ- and δ-opioid receptors). Initial profiling of 5 revealed less than 50% displacement of radioligand binding to as μ- and δ-opioid receptors (Psychoactive Drug Screening Program and UNC-CH). Some embodiments, the compounds disclosed herein, exhibit a selective binding activity towards the KOR that is at least about 2-fold, about 3-fold, about 4-fold, or at least about 5-fold more potent compared to its binding activity towards other opioid receptors.

In addition, a biased ligand refers to compounds that display an increased potency and or efficacy for one signaling pathway over another as compared to a reference agonist (in this case U69,593). Particular examples in the report display a preference for initiating GTPyS binding upon receptor activation relative to β-arrestin recruitment. In some cases, the preference for initiating GTPyS binding is greater than the ability to inhibit forskolin-stimulated adenylyl cyclase as measured by cAMP accumulation as well. In some embodiments, the compounds disclosed herein are, by simplistic comparison of IC50 values more potent for GTPyS activation over β-arrestin recruitment, relative to that observed for U69,593.

In some embodiments, the compound of Formulae (A), (I), (II), or (III) is a compound provided herein, or a pharmaceutically acceptable salt thereof. In some embodiments, the compound of Formula (A) is a compound provided herein, or a pharmaceutically acceptable salt thereof. In some embodiments, the compound of Formula (I) is a compound provided herein, or a pharmaceutically acceptable salt thereof. In some embodiments, the compound of Formula (II) is a compound provided herein, or a pharmaceutically acceptable salt thereof. In some embodiments, the compound of Formula (III) is a compound provided herein, or a pharmaceutically acceptable salt thereof.

In some embodiments, the compound of Formulae (A), (I), (II), and (III) is a compound provided in a Table herein, or a pharmaceutically acceptable salt thereof. In some embodiments, the compound of Formula (A) is a compound provided in a Table herein (e.g., Tables 1-8), or a pharmaceutically acceptable salt thereof. In some embodiments, the compound of Formula (I) is a compound provided in a Table herein (e.g., Tables 1-7), or a pharmaceutically acceptable salt thereof. In some embodiments, the compound of Formula (II) is a compound provided in a Table herein (e.g., Tables 1-7), or a pharmaceutically acceptable salt thereof. In some embodiments, the compound of Formula (III) is a compound provided in a Table herein (e.g., Tables 1-7), or a pharmaceutically acceptable salt thereof. A compound of any one of Formula (I), (II), and (III) may be selected from the compound listed in the following Tables. Compounds of Formulae (A), (I), (II), and (III) that are not listed in the following Tables are also within the scope herein.

Table 1 below shows compounds with various linkers.

TABLE 1
Structure-Activity Relationship Investigation of Linker Length

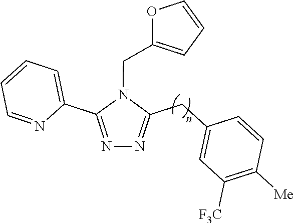

| | | GTPγS^{a} | | β-arrestin2^{b} | | | |
|---|---|---|---|---|---|---|---|
| | | EC₅₀ | Eₘₐₓ | EC₅₀ | Eₘₐₓ | | Microsomal |
| Compound No. | n | (nM) | (% U60) | (nM) | (% U60) | Bis^{c} | stability |
| U69,693 | | 43 | 100 | 55 | 100 | 1 | ND |
| (reference) | | | | | | | |
| 1 | 1 | nc | nc | nc | nc | nc | 0.9 |
| 2 | 2 | 21 | 90 | 788 | 119 | 11 | 0.9 |
| 3 | 3 | 812 | 74 | nc | nc | nc | 1.6 |

Pharmacological parameters from the GTPγS binding assay and the βarrestin2 enayme fragment complementation assay as described in Zhou et al., 2013 (22). EC₅₀ and Emax were derived from a 3 parameter nonlinear regression analysis of a concentration response analysis and curve fitting using Graphpad Prism (9.0) software. Data are presented in reference to U69,593 which was assayed along side each compound tested and was used to define the maximal response (100% Emax).
Bias analysis was performed by the operational model curve fitting analysis using Graphpad Prism (9.0) software using the global parameter fitting of the averaged data. Bias factors are further calculated as the 10\hat{ }\hat{ }ΔΔ(logτ/K_{A})_{Gprotein Barr2} with U69,593 serving as the reference agonist, as described in Zhou et al., (22). Bias analysis was not applied for compounds with single experiments or if the EC₅₀ in GTPγS binding was greater than 150 nM. ~ did not plateau at 100%, EC₅₀ estimated.
Bias factor was only calculated for n ≥ 3 or more and if the EC50 in GTP is less than 150 nM. Analysis is based on global parameter fitting of the replicates; vlaues may change as individual replicates are individually fit to the analysis nc: did not converge to nonlinear regression;
*maximum stimulation observed at 10 μM for compounds that did not reach plateau;
ND—not determined.
Microsomal stability measures were determined in preparations of mouse hepatic microsomes as previously described (Zhou et al., 2013, 22)).

Compound 2 exhibits good activity with respect to G-protein activation. Interestingly, the two-carbon linker is shorter compared to the sulfur-containing side chain of Triazole 1.1 as the length of a C—S bond is longer than the length of a C—C bond. With respect to microsomal stability, Compound 2 is similar to that of Triazole 1.1, 0.9 and 1.1 min respectively.

Next, Table 2 shows compounds with various Ar₂ substituents.

TABLE 2
Structure-Activity Relationship Investigation of Ar₂

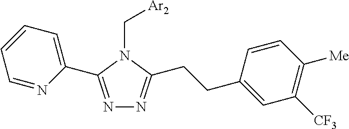

| | | GTPγS | | ß-arrestin | | | |
|---|---|---|---|---|---|---|---|
| Compound | | EC₅₀ | Eₘₐₓ | EC₅₀ | Eₘₐₓ | | Microsomal |
| No. | Ar₂ | (nM) | (% U60) | (nM) | (% U60) | Bias | Stability (min) |
| 2 | 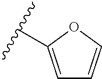 [SMILES: *c1ccco1] | 21 | 90 | 788 | 119 | 11 | 0.9 |
| 5 | 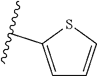 [SMILES: *c1cccs1] | 11 | 95 | 624 | 110 | 28 | 01.4 |
| 6 | 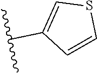 [SMILES: *c1ccsc1] | 12 | 85 | 813 | 125 | 9 | 1.0 |
| 7 | 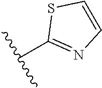 [SMILES: *c1nccs1] | 51 | 63 | 3566 | 82 | 13 | 2.1 |
| 8 | 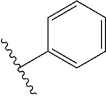 [SMILES: *c1ccccc1] | 83 | 88 | 6342 | 180 | 7 | 0.9 |
| 10 | 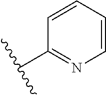 [SMILES: *c1ccccn1] | 175 | 784 | 7690 | 125 | 7 | 1.0 |
| 11 | 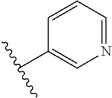 [SMILES: *c1cccnc1] | 70 | 90 | 479 | 105 | 2 | 1.7 |
| 12 | 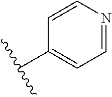 [SMILES: *c1ccncc1] | n.c. | 48* | n.c. | 12* | ND | 0.9 |
| 13 | 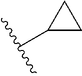 [SMILES: *C1CC1] | 213 | 101 | 6221 | 122 | 25 | 0.9 |
| 5a | 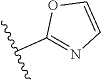 [SMILES: *c1ncco1] | 38 | 97 | 532 | 88 | ND | ND |

Pharmacological parameters from the GTPγS binding assay and the βarrestin2 enzyme fragment complementation assay as described in Zhou et al., 2013 (22). EC₅₀ and Emax were derived from a 3 parameter nonlinear regression analysis of a concentration response analysis and curve fitting using Graphpad Prism (9.0) software. Data are presented in reference to U69,593 which was assayed along side each compound tested and was used to define the maximal response (100% Emax). Bias analysis
was performed by the operational model curve fitting analysis using Graphpad Prism (9.0) software using the global parameter fitting of the averaged data. Bias factors are further calculated as the 10\hat{ }ΔΔ(logτ/K_{A})_{Gprotein-Barr2} with U69,593 serving as the reference agonist, as described in Zhou et al., (22). Bias analysis was not applied for compounds with single experiments or if the EC₅₀ in GTPγS binding was greater than 150 nM. ~ did not plateau at 100%, EC₅₀ estimated. Bias factor was only
calculated for n ≥ 3 or more and if the EC50 in GTP is less than 150 nM. Anaylsis is based on global parameter fitting of the replicates; values may change as individual replicates are individually fit to the analysis nc: did not converge to nonlinear regression;
*maximum stimulation observed at 10 μM for compounds that did not reach plateau;
ND—not determined.
Microsomal stability measures were determined in preparations of mouse hepatic microsomes as previously described (Zhou et al., 2013, (22)).

In Table 2 compounds with aromatic substitutions on the 4-position of the triazole 1.1 are shown, which are structurally different from previous SAR studies.^{21,27,28} First, the furan moiety was replaced with other 5-membered aromatic heterocycles. Both thiophene and thiazole replacements led to compounds of similar potency (Compounds 5 and 7). Next, the size of the aromatic ring was investigated by increasing to a benzyl substitution which led to a slight decrease in potency (Compound 8). However, when the —CH₂ linkage was removed

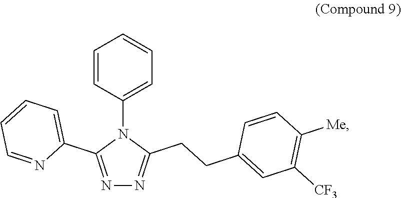 [SMILES: Cc1ccc(CCc2nnc(-c3ccccn3)n2-c2ccccc2)cc1C(F)(F)F]

potency was completely lost indicating the methylene linker is beneficial for positioning of the aromatic ring. Pyridine rings are also tolerated (Compounds 10, 11 and 12). Finally, due to the frequency of cyclopropane substituents in KOR agonists,³¹ However, Compound 13 did not exhibit any increase in potency.

Next, Table 3 shows compounds with various Ani substituents.

TABLE 3
Structure-Activity Relationship Investigation of Ar₁

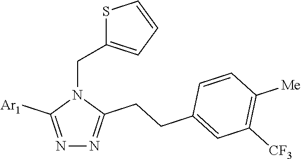

| | | GTPγS | | ß-arrestin | | | |
|---|---|---|---|---|---|---|---|
| Compound | | EC₅₀ | Eₘₐₓ | EC₅₀ | Eₘₐₓ | | Microsomal |
| No. | Ar₁ | (nM) | (% U60) | (nM) | (% U60) | Bias | Stability (min) |
| 14 | 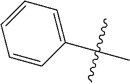 [SMILES: *c1ccccc1] | 406 | 89 | >10000 | ~100 | ND | 1.8 |
| 5 | 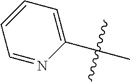 [SMILES: *c1ccccn1] | 11 | 95 | 624 | 110 | 28 | 1.4 |
| 15 | 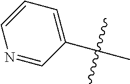 [SMILES: *c1cccnc1] | 1883 | 85 | n.c. | 14* | n.c. | 1.0 |
| 16 | 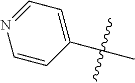 [SMILES: *c1ccncc1] | n.c. | 19* | n.c. | 0* | ND | ND |
| 17 | 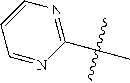 [SMILES: *c1ncccn1] | 57 | 85 | 2183 | 150 | 6 | 0.8 |
| 18 | 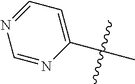 [SMILES: *c1ccncn1] | 991 | 82 | >10000 | 66* | ND | 1.1 |
| 19 | 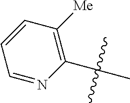 [SMILES: *c1ncccc1C] | 13 | 88 | 1549 | 117 | 30 | 1.1 |
| 20 | 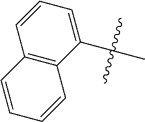 [SMILES: *c1cccc2ccccc12] | 429 | 86 | >10000 | 76* | ND | 0.9 |
| 21 | 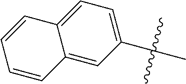 [SMILES: *c1ccc2ccccc2c1] | 2959 | 64 | n.c. | 16* | ND | 1.3 |
| 22 | 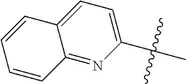 [SMILES: *c1ccc2ccccc2n1] | 872 | 80 | >10000 | ~100 | ND | 2.2 |
| 23 | 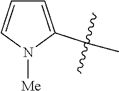 [SMILES: *c1cccn1C] | 36 | 94 | 1319 | 105 | 25 | 3.1 |

Pharmacological parameters from the GTPγS binding assay and the βarrestin2 enzyme fragment complementation assay as described in Zhou et al., 2013 (22). EC₅₀ and Emax were derived from a 3 parameter nonlinear regression analysis of a concentration response analysis and curve fitting using Graphpad Prism (9.0) software. Data are presented in reference to U69,593 which was assayed along side each compound tested and was used to define the maximal response (100% Emax). Bias analysis
was performed by the operational model curve fitting analysis using Graphpad Prism (9.0) software using the global parameter fitting of the averaged data. Bias factors are further calculated as the 10\hat{ }ΔΔ(logτ/K_{A})_{Gprotein-Barr2} with U69,593 serving as the reference agonist, as described in Zhou et al., (22). Bias analysis was not applied for compounds with single experiments or if the EC50 in GTPγS binding was greater than 150 nM. ~ did not plateau at 100%, EC₅₀ estimated. Bias factor was only
calculated for n ≥ 3 or more and if the EC50 in GTP is less than 150 nM. Analysis is based on global parameter fitting of the replicates for some of the compounds; values may change as individual replicates are individually fit to the analysis nc: did not converge to nonlinear regression;
*maximum stimulation observed at 10 μM for compounds that did not reach plateau;
ND—not determined.
Microsomal stability measures were determined in preparations of mouse hepatic microsomes as previously described (Zhou et al., 2013, (22)).

Table 3 contains compounds where the 5-position of the triazole scaffold was examined. Here Compounds 15, 20 and 24 exhibited good activity. Extension of the linker connected to naphthyl substitutions is less well tolerated, though this is likely due to the lack of a nitrogen in the 2-position, such as in a 2-pyridyl heteroaryl. Finally, 5-membered rings with a nitrogen in the 2-position are also tolerated.

Next, Table 4 shows compounds with various Ar₃ substituents.

TABLE 4
Structure-Activity Relationship Investigation of Ar₃

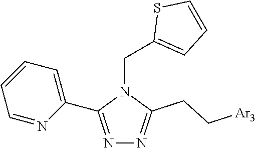

| | | GTPγS^{a} | | ß-arrestin | | | |
|---|---|---|---|---|---|---|---|
| Compound | | EC₅₀ | Eₘₐₓ | EC₅₀ | Emax | | Microsomal |
| No. | Ar₃ | (nM) | (% U60) | (nM) | (% U60) | Bias | Stability (min) |
| 24 | 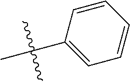 [SMILES: *c1ccccc1] | 926 | 76 | >10000 | 31 | ND | 5.5 |
| 25 | 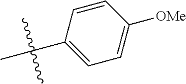 [SMILES: *c1ccc(OC)cc1] | 122 | 96 | >10000 | 124 | ND | 5.6 |
| 26 | 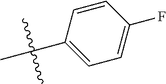 [SMILES: *c1ccc(F)cc1] | 288 | 92 | 3024 | 75 | 4 | 3.1 |
| 27 | 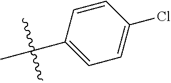 [SMILES: *c1ccc(Cl)cc1] | 35 | 93 | 3735 | 102 | 25 | 3.3 |
| 28 | 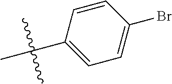 [SMILES: *c1ccc(Br)cc1] | 20 | 96 | 963 | 106 | 29 | 4.6 |
| 29 | 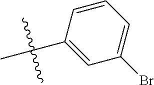 [SMILES: *c1cccc(Br)c1] | 51 | 89 | 5019 | 114 | 22 | 3.1 |
| 30 | 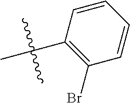 [SMILES: *c1ccccc1Br] | 358 | 84 | >10000 | 53* | ND | 1.7 |
| 31 | 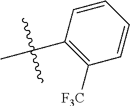 [SMILES: *c1ccccc1C(F)(F)F] | 429 | 84 | >10000 | 44 | ND | 3.0 |
| 5 | 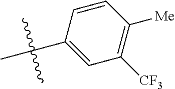 [SMILES: *c1ccc(C)c(C(F)(F)F)c1] | 11 | 95 | 624 | 110 | 28 | 1.4 |
| 32 | 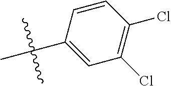 [SMILES: *c1ccc(Cl)c(Cl)c1] | 5 | 101 | 319 | 107 | 70 | 1.5 |
| 33 | 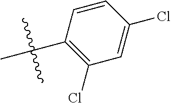 [SMILES: *c1ccc(Cl)cc1Cl] | 32 | 91 | 5161 | 154 | 77 | 2.1 |
| 34 | 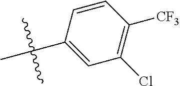 [SMILES: *c1ccc(C(F)(F)F)c(Cl)c1] | 1.4 | 95 | 127 | 116 | 67 | 1.3 |
| 35 | 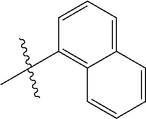 [SMILES: *c1cccc2ccccc12] | 184 | 91 | >10000 | 67 | ND | 1 |
| 36 | 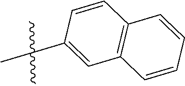 [SMILES: *c1ccc2ccccc2c1] | 12 | 92 | 896 | 154 | 13 | 2.4 |
| 39a | 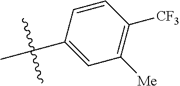 [SMILES: *c1ccc(C(F)(F)F)c(C)c1] | 3.1 | ND | ND | ND | ND | ND |

Pharmacological parameters from the GTPγS binding assay and the βarrestin2 enzyme fragment complementation assay as described in Zhou et al., 2013 (22). EC₅₀ and Emax were derived from a 3 parameter nonlinear regression analysis of a concentration response analysis and curve fitting using Graphpad Prism (9.0) software. Data are presented in reference to U69,593 which was assayed along side each compound tested and was used to define the maximal response (100% Emax). Bias analysis was performed by the
operational model curve fitting analysis using Graphpad Prism (9.0) software using the global parameter fitting of the averaged data. Bias factors are further calculated as the 10\hat{ }ΔΔ (logτ/K_{A})_{Gprotein-Barr2} with U69,593 serving as the reference agonist, as described in Zhou et al., (22). Bias analysis was not applied for compounds with single experiments or if the EC₅₀ in GTPγS binding was greater than 150 nM. ~ did not plateau at 100%, EC₅₀ estimated. Bias factor was only calculated for n ≥ 3 or more and if the EC50
in GTP is less than 150 nM. Analysis is based on global parameter fitting of the replicates; values may change as individual replicates are individually fit to the analysis nc: did not converge to nonlinear regression;
*maximum stimulation observed at 10 μM for compounds that did not reach plateau;
ND—not determined.
Microsomal stability measures were determined in preparations of mouse hepatic microsomes as previously described (Zhou et al., 2013, 22)).

Table 4 shows compounds with various aromatic rings having substitutions for Ar₃.

As a first, it should be noted that when Ar₃ is a substituted aromatic ring, better activity is observed compared to compounds having unsubstituted aromatic rings for Ar₃. Although electron donating groups are tolerated as substituents on the aromatic ring, electron withdrawing groups appear to be preferred. For mono-substituted aromatic rings, substitution at the 4-position appears to be preferred. 3,4 Di-substituted aromatic rings exhibit increased potency compared to mono-substituted aromatic rings.

Next, Table 5 shows compounds with various Linkers (L).

TABLE 5
Structure-Activity Relationship Investigation of Carbon Linker (L)

| | | GTPγS | | ß-arrestin2 | | | Microsomal |
|---|---|---|---|---|---|---|---|
| Compound | | EC₅₀ | Eₘₐₓ | EC₅₀ | Eₘₐₓ | | Stability |
| No. | Structure | (nM) | (% U60) | (nM) | (% U60) | Bias | (min) |
| 37 | 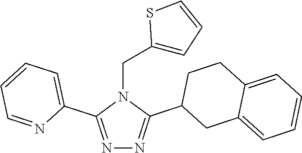 [SMILES: c1ccc(-c2nnc(C3CCc4ccccc4C3)n2Cc2cccs2)nc1] | n.c. | 9* | n.c. | 0* | ND | 13.1 |
| 38 | 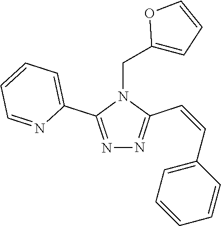 [SMILES: C(=C/c1nnc(-c2ccccn2)n1Cc1ccco1)/c1ccccc1] | n.c. | 3* | n.c. | 0* | ND | 19.4 |
| 39 | 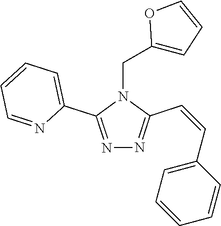 [SMILES: C(=C/c1nnc(-c2ccccn2)n1Cc1ccco1)/c1ccccc1] | 1795 | 80 | n.c. | 19* | ND | 12.6 |
| 40 | 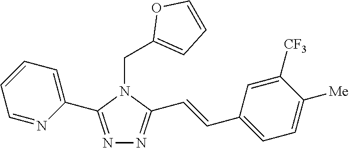 [SMILES: Cc1ccc(/C=C/c2nnc(-c3ccccn3)n2Cc2ccco2)cc1C(F)(F)F] | 1598 | 97 | 7199 | 54 | ND | 18 |
| 41 | 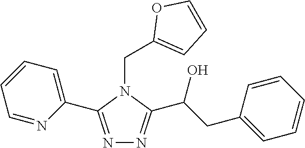 [SMILES: OC(Cc1ccccc1)c1nnc(-c2ccccn2)n1Cc1ccco1] | n.c. | 22* | n.c. | 0* | ND | 17.4 |
| 42 | 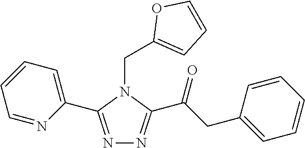 [SMILES: O=C(Cc1ccccc1)c1nnc(-c2ccccn2)n1Cc1ccco1] | n.c. | 4* | n.c. | 2 | ND | 8.1 |
| 43 | 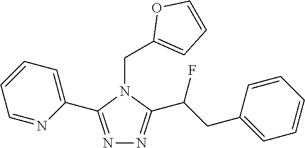 [SMILES: FC(Cc1ccccc1)c1nnc(-c2ccccn2)n1Cc1ccco1] | 3825 | 49 | >10000 | 7* | ND | 9.1 |
| 44 | 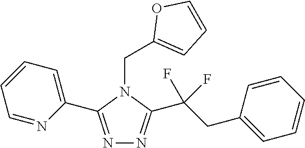 [SMILES: FC(F)(Cc1ccccc1)c1nnc(-c2ccccn2)n1Cc1ccco1] | | | | | | |
| 45 | 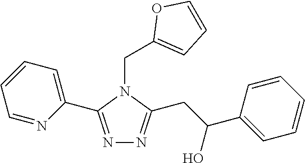 [SMILES: OC(Cc1nnc(-c2ccccn2)n1Cc1ccco1)c1ccccc1] | n.c. | n.c. | n.c. | n.c. | ND | 20.7 |
| 46 | 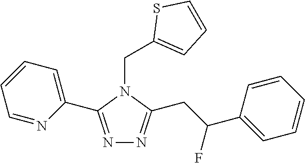 [SMILES: FC(Cc1nnc(-c2ccccn2)n1Cc1cccs1)c1ccccc1] | 1063 | 72* | >10000 | 64* | ND | 69.7 |

Pharmacological parameters from the GTPγS binding assay and the βarrestin2 enzyme fragment complementation assay as described in Zhou et al., 2013 (22). EC₅₀ and Emax were derived from a 3 parameter nonlinear regression analysis of a concentration response analysis and curve fitting using Graphpad Prism (9.0) software. Data are presented in reference to U69,593 which was assayed along side each compound tested and was used to define the maximal response (100% Emax). Bias analysis
was performed by the operational model curve fitting analysis using Graphpad Prism (9.0) software using the global parameter fitting of the averaged data. Bias factors are further calculated as the 10\hat{ }ΔΔ(logτ/K_{A})_{Gprotein-Barr2} with U69,593 serving as the reference agonist, as described in Zhou et al., (22). Bias analysis was not applied for compounds with single experiments or if the EC₅₀ in GTPγS binding was greater than 150 nM. ~ did not plateau at 100%, EC₅₀ estimated. Bias factor was only
calculated for n ≥ 3 or more and if the EC50 in GTP is less than 150 nM. Analysis is based on global parameter fitting of the replicates; values may change as individual replicates are individually fit to the analysis nc: did not converge to nonlinear regression;
*maximum stimulation observed at 10 μM for compounds that did not reach plateau;
ND—not determined.
Microsomal stability measures were determined in preparations of mouse hepatic microsomes as previously described (Zhou et al., 2013, (22)).

A variety of compounds in which linker (L) was modified were prepared and screened. As a first, Compounds such as 39 where rotation around linker L is restricted resulted in a loss of activity. Next, several compounds were prepared in which the linker had varying degrees of unsaturation. In all cases, a loss in potency was observed (Compounds 40, 41, and 42). Next, compounds containing alpha alcohols, alpha ketones, and beta alcohols are completely inactive (Compounds 43, 44, and 47). Finally, Compounds with a fluorinated linker were prepared (Compounds 46 and 48) as well to see if an improvement in metabolic stability could be observed as fluorine is well-known to slow down metabolism.³²

Next, Table 6 shows compounds with various trans-alkene Linkers (L).

TABLE 6
Structure-Activity Relationship Investigation of Trans-Alkene Linkers (L)

| | | GTPγS^{a} | | ß-arrestinb | | | Microsomal |
|---|---|---|---|---|---|---|---|
| Compound | | EC₅₀ | Eₘₐₓ | | Eₘₐₓ | | Stability |
| No. | Structure | (nM) | (% U60) | EC₅₀ (nM) | (% U60) | Bias^{c} | (min)^{d} |
| 40 | 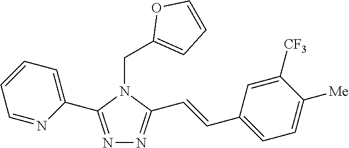 [SMILES: Cc1ccc(/C=C/c2nnc(-c3ccccn3)n2Cc2ccco2)cc1C(F)(F)F] | 1598 | 97 | 7199 | 54 | 5 | 18 |
| 47 | 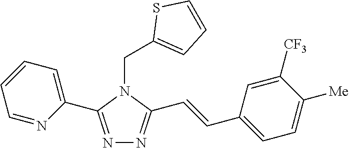 [SMILES: Cc1ccc(/C=C/c2nnc(-c3ccccn3)n2Cc2cccs2)cc1C(F)(F)F] | 667 | 83 | 20600 | 36* | ND | 22.0 |
| 48 | 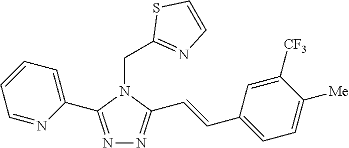 [SMILES: Cc1ccc(/C=C/c2nnc(-c3ccccn3)n2Cc2nccs2)cc1C(F)(F)F] | 767 | 82 | n.c. | 51* | ND | 19.1 |
| 49 | 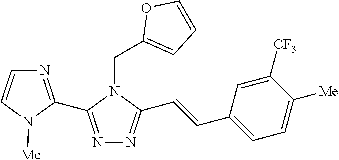 [SMILES: Cc1ccc(/C=C/c2nnc(-c3nccn3C)n2Cc2ccco2)cc1C(F)(F)F] | 29100 | 76 | n.c. | 17* | ND | 14.1 |
| 50 | 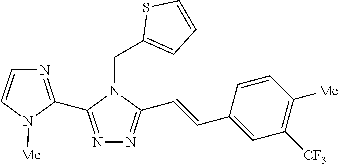 [SMILES: Cc1ccc(/C=C/c2nnc(-c3nccn3C)n2Cc2cccs2)cc1C(F)(F)F] | 1008 | 77 | 55800 | 38* | ND | 12.2 |
| 51 | 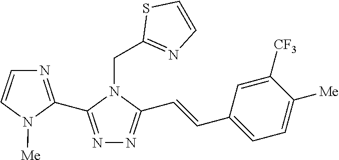 [SMILES: Cc1ccc(/C=C/c2nnc(-c3nccn3C)n2Cc2nccs2)cc1C(F)(F)F] | 309 | 70 | 1683 | 34 | 4 | 14.5 |
| 42 (UNC9008) | 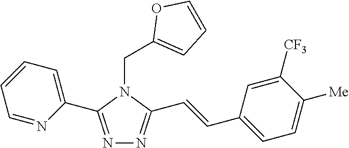 [SMILES: Cc1ccc(/C=C/c2nnc(-c3ccccn3)n2Cc2ccco2)cc1C(F)(F)F] | 1600 | 96.5 | 51300 | 119 | 3.5 | |
| 49 (UNC9009) | 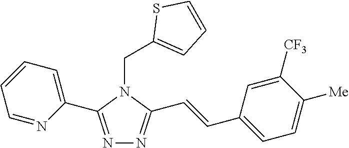 [SMILES: Cc1ccc(/C=C/c2nnc(-c3ccccn3)n2Cc2cccs2)cc1C(F)(F)F] | 667 | 83.2 | 20600 | 36.4 (% max at 10 uM) | 6.7 | |
| 50 (UNC9078) | 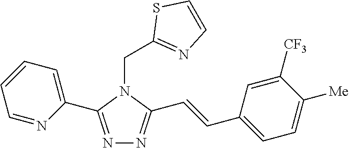 [SMILES: Cc1ccc(/C=C/c2nnc(-c3ccccn3)n2Cc2nccs2)cc1C(F)(F)F] | 767 | 81.6 | n.c. | 51.6 (% max at 10 uM) | nc | 10.1 |
| 51 (UNC9012) | 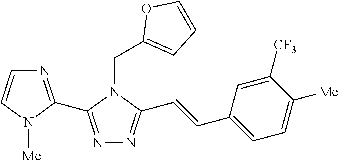 [SMILES: Cc1ccc(/C=C/c2nnc(-c3nccn3C)n2Cc2ccco2)cc1C(F)(F)F] | 29100 | 76.1 | n.c. | 17.3 (% max at 10 uM) | nc | |
| 52 (UNC9166) | 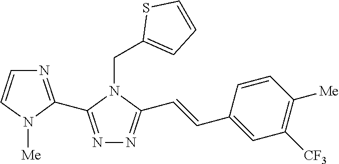 [SMILES: Cc1ccc(/C=C/c2nnc(-c3nccn3C)n2Cc2cccs2)cc1C(F)(F)F] | 349 | 74.2 | 55800 | 37.7 (% max at 10 uM) | | 12.2 |
| 53 (UNC9081) | 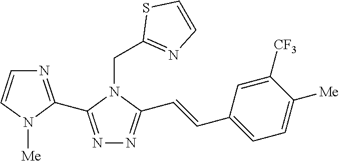 [SMILES: Cc1ccc(/C=C/c2nnc(-c3nccn3C)n2Cc2nccs2)cc1C(F)(F)F] | 309 | 70.5 | 1570 | 25.9 (% max at 10 uM) | 10.4 | 14.5 |

Pharmacological parameters from the GTPγS binding assay and the βarrestin2 enzyme fragment complementation assay as described in Zhou et al., 2013 (22). EC₅₀ and Emax were derived from a 3 parameter nonlinear regression analysis of a concentration response analysis and curve fitting using Graphpad Prism (9.0) software. Data are presented in reference to U69,593 which was assayed along side each compound tested and was used to define the maximal response (100% Emax). Bias analysis
was performed by the operational model curve fitting analysis using Graphpad Prism (9.0) software using the global parameter fitting of the averaged data. Bias factors are further calculated as the 10\hat{ }ΔΔ(logτ/K_{A})_{Gprotein-Barr2} with U69,593 serving as the reference agonist, as described in Zhou et al., (22). Bias analysis was not applied for compounds with single experiments or if the EC₅₀ in GTPγS binding was greater than 150 nM. ~ did not plateau at 100%, EC₅₀ estimated. Bias factor was only
calculated for n ≥ 3 or more and if the EC50 in GTP is less than 150 nM. Analysis is based on global parameter fitting of the replicates; values may change as individual replicates are individually fit to the analysis nc: did not converge to nonlinear regression;
*maximum stimulation observed at 10 μM for compounds that did not reach plateau;
ND—not determined.
Microsomal stability measures were determined in preparations of mouse hepatic microsomes as previously described (Zhou et al., 2013, (22)).

Given the increased metabolic stability of the trans-olefin containing Compound 42, additional compounds were further investigated with this type of linker. In all cases, a loss in potency of these compounds was observed. However, for those tested, an increase in metabolic stability was noted. For example, despite the loss in potency, Compound 54 appears to be a weak partial agonist with an Emax of about 26%. Partial agonists of KOR have been of interest due to their potential to treat opioid use disorder.^{33,34}

Next, Table 7 shows some additional compounds that were prepared.

TABLE 7
Additional Analogues

| | | GTPγS^{a} | | ß-arrestin^{b} | | | Microsoma |
|---|---|---|---|---|---|---|---|
| | | EC₅₀ | Eₘₐₓ | EC₅₀ | Eₘₐₓ | | 1 Stability |
| Compound No. | Structure | (nM) | (% U60) | (nM) | (% U60) | Bias^{c} | (min)^{d} |
| 52 | 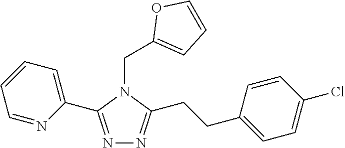 [SMILES: Clc1ccc(CCc2nnc(-c3ccccn3)n2Cc2ccco2)cc1] | 99 | 84 | 4531 | 191 | 9 | 3.3 |
| 53 | 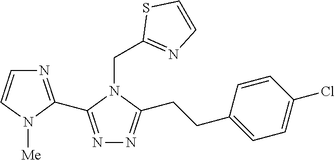 [SMILES: Cn1ccnc1-c1nnc(CCc2ccc(Cl)cc2)n1Cc1nccs1] | 2252 | 94 | 5018 | 81 | ND | 2.4 |
| 54 | 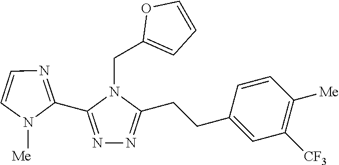 [SMILES: Cc1ccc(CCc2nnc(-c3nccn3C)n2Cc2ccco2)cc1C(F)(F)F] | 69 | 92 | 788 | 112 | 8 | 1.6 |
| 55 | 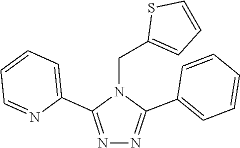 [SMILES: c1ccc(-c2nnc(-c3ccccn3)n2Cc2cccs2)cc1] | n.c. | n.c. | n.c. | n.c. | ND | ND |
| 56 | 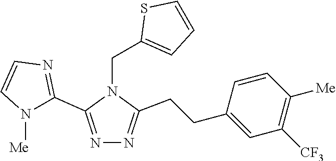 [SMILES: Cc1ccc(CCc2nnc(-c3nccn3C)n2Cc2cccs2)cc1C(F)(F)F] | 95 | 105 | 888 | 98 | 12 | ND |

Pharmacological parameters from the GTPγS binding assay and the βarrestin2 enzyme fragment complementation assay as described in Zhou et al., 2013 (22). EC₅₀ and Emax were derived from a 3 parameter nonlinear regression analysis of a concentration response analysis and curve fitting using Graphpad Prism (9.0) software. Data are presented in reference to U69,593 which was assayed along side each compound tested and was used to define the maximal response (100% Emax). Bias analysis was performed by the
operational model curve fitting analysis using Graphpad Prism (9.0) software using the global parameter fitting of the averaged data. Bias factors are further calculated as the 10\hat{ }ΔΔ(logτ/K_{A})_{Gprotein-Barr2} with U69,593 serving as the reference agonist, as described in Zhou et al., (22). Bias analysis was not applied for compounds with single experiments or if the EC₅₀ in GTPγS binding was greater than 150 nM. ~ did not plateau at 100%, EC₅₀ estimated. Bias factor was only calculated for n ≥ 3 or more and if the EC50 in
GTP is less than 150 nM. analysis is based on global parameter fitting of the replicates; values may change as individual replicates are individually fit to the analysis nc: did not converge to nonlinear regression;
*maximum stimulation observed at 10 μM for compounds that did not reach plateau;
ND—not determined.
Microsomal stability measures were determined in preparations of mouse hepatic microsomes as previously described (Zhou et al., 2013, 22)).

Next, Table 8 shows compounds where the triazole core is missing.

TABLE 8
Structure Activity Relationships for Deconstructed Triazoles

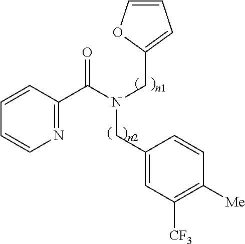

| Com- | | | GTPγS^{a} | | ß-arrestin^{b} | | | Microsomal |
|---|---|---|---|---|---|---|---|---|
| pound | | | EC₅₀ | Eₘₐₓ | EC₅₀ | Eₘₐₓ | | Stability |
| No. | n1 | n2 | (nM) | (% U60) | (nM) | (% U60) | Bias^{c} | (min)^{d} |
| 57 | 1 | 1 | 368 | 92 | 3134 | 87 | 10 | 1.8 |
| 58 | 1 | 2 | 476 | 94 | 5334 | 112 | 7 | 0.9 |
| 59 | 1 | 3 | 219 | 89 | 3205 | 92 | 14 | 1.8 |
| 60 | 2 | 1 | 124 | 93 | 5511 | 88* | ND | 1.8 |
| 61 | 2 | 2 | 1541 | 78 | >10000 | 41* | ND | 0.9 |
| 62 | 2 | 3 | 1508 | 97 | >10000 | 34* | ND | 1.0 |
| 63 | 3 | 1 | 670 | 100 | >10000 | 34* | ND | 1.1 |
| 64 | 3 | 3 | 3058 | 88 | >10000 | 26* | ND | 1.0 |

Pharmacological parameters from the GTPγS binding assay and the βarrestin2 enzyme fragment complementation assay as described in Zhou et al., 2013 (22). EC₅₀ and Emax were derived from a 3 parameter nonlinear regression analysis of a concentration response analysis and curve fitting using Graphpad Prism (9.0) software. Data are presented in reference to U69,593 which was assayed along side each compound tested and was used to define the maximal response (100% Emax). Bias analysis
was performed by the operational model curve fitting analysis using Graphpad Prism (9.0) software using the global parameter fitting of the averaged data. Bias factors are further calculated as the 10\hat{ }ΔΔ(logτ/K_{A})_{Gprotein-Barr2} with U69,593 serving as the reference agonist, as described in Zhou et al., (22). Bias analysis was not applied for compounds with single experiments or if the EC₅₀ in GTPγS binding was greater than 150 nM. ~ did not plateau at 100%, EC₅₀ estimated. Bias factor was only
calculated for n ≥ 3 or more and if the EC50 in GTP is less than 150 nM. analysis is based on global parameter fitting of the replicates; values may change as individual replicates are individually fit to the analysis nc: did not converge to nonlinear regression;
*maximum stimulation observed at 10 μM for compounds that did not reach plateau;
ND—not determined.
Microsomal stability measures were determined in preparations of mouse hepatic microsomes as previsously described (Zhou et al., 2013, 22)).

Table 7 shows the importance of the role of the triazole core with respect to KOR activity. Various compounds were prepared with a deconstructed triazole scaffold. Although these deconstructed compounds retain agonist activity, they are significantly less potent than the triazole compounds as disclosed herein, indicating that the triazole core plays an important role in the activity of the disclosed triazole compounds. One hypothesis for this observation is that the triazole core orients the three substituents, Ar₁, Ar₂, and Ar₃, in the binding pocket to maximize binding interactions to the KOR.

Table 9 shows additional compounds.

TABLE 9
Additional Compounds of Formula (A)

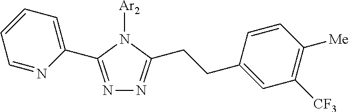

| | | GTPγS | | ß-arrestin | | | |
|---|---|---|---|---|---|---|---|
| Compound | | EC₅₀ | Eₘₐₓ | EC₅₀ | Eₘₐₓ | | Microsomal |
| No. | Ar₂ | (nM) | (% U60) | (nM) | (% U60) | Bias | Stability (min) |
| 8a | 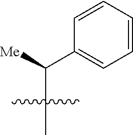 [SMILES: *[C@@H](C)c1ccccc1] | ND | ND | ND | ND | ND | ND |
| 9a | 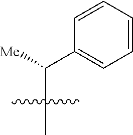 [SMILES: *[C@H](C)c1ccccc1] | ND | ND | ND | ND | ND | ND |
| 11a | 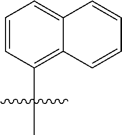 [SMILES: *c1cccc2ccccc12] | 2646 | 89 | nc | 51 | ND | ND |
| 12a | 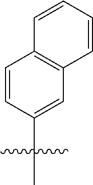 [SMILES: *c1ccc2ccccc2c1] | 10283 | 140 | nc | 41 | ND | ND |
| 13a | 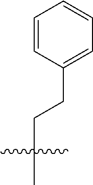 [SMILES: *CCc1ccccc1] | 1943 | 96 | nc | 78 | ND | ND |
| 17a | 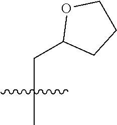 [SMILES: *CC1CCCO1] | 104 | 81 | 5432 | 121 | ND | ND |

Pharmacological parameters from the GTPγS binding assay and the βarrestin2 enzyme fragment complementation assay as described in Zhou et al., 2013 (22). EC₅₀ and Emax were derived from a 3 parameter nonlinear regression analysis of a concentration response analysis and curve fitting using Graphpad Prism (9.0) software. Data are presented in reference to U69,593 which was assayed along side each compound tested and was used to define the maximal response (100% Emax). Bias analysis
was performed by the operational model curve fitting analysis using Graphpad Prism (9.0) software using the global parameter fitting of the averaged data. Bias factors are further calculated as the 10\hat{ }ΔΔ(logτ/K_{A})_{Gprotein-Barr2} with U69,593 serving as the reference agonist, as described in Zhou et al., (22). Bias analysis was not applied for compounds with single experiments or if the EC₅₀ in GTPγS binding was greater than 150 nM. ~ did not plateau at 100%, EC₅₀ estimated. Bias factor was only
calculated for n ≥ 3 or more and if the EC50 in GTP is less than 150 nM. analysis is based on global parameter fitting of the replicates; values may change as individual replicates are individually fit to the analysis nc: did not converge to nonlinear regression;
*maximum stimulation observed at 10 μM for compounds that did not reach plateau;
ND—not determined.
Microsomal stability measures were determined in preparations of mouse hepatic microsomes as previously described (Zhou et al., 2013, (22)).

### C. Methods of Preparing

The current disclosure is also directed to any methods for preparing the compounds disclosed herein. A skilled artisan would be aware that such preparative methods can vary. The compounds disclosed herein may be synthesized by synthetic routes that include processes analogous to those well-known in the chemical arts, particularly in light of the description contained herein, and those for heterocycles such as triazoles described in: Comprehensive Heterocyclic Chemistry II, Editors Katritzky and Rees, Elsevier, 1997, e.g., Volume 3; Liebigs Annalen der Chemie, (9):1910-16, (1985); Helvetica Chimica Acta, 41:1052-60, (1958); Arzneimittel-Forschung, 40(12):1328-31, (1990), each of which are expressly incorporated by reference. Starting materials are generally available from commercial sources or are readily prepared using methods well known to those skilled in the art (e.g., prepared by methods generally described in Louis F. Fieser and Mary Fieser, Reagents/or Organic Synthesis, v. 1-23, Wiley, N.Y. (1967-2006 ed.), or Beilsteins Handbuch der organischen Chemie, 4, Aufl. ed. Springer-Verlag, Berlin, including supplements (also available via the Beilstein online database)).

Exemplary methods are described in detail further below, i.e., the preparation of compounds of Formula (I), wherein L is —(CH₂)ᵣ—. Such methods comprise:
contacting intermediate A

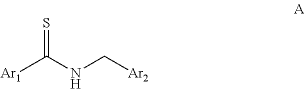

with an intermediate B

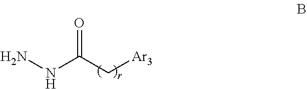

to render a compound of Formula (I), wherein L is —(CH₂)ᵣ—

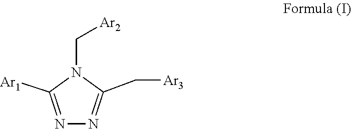

Intermediate A can be synthesized from precursors A.1 and A.2, and intermediate B can be synthesized from precursor B.1, according to the general scheme shown below:

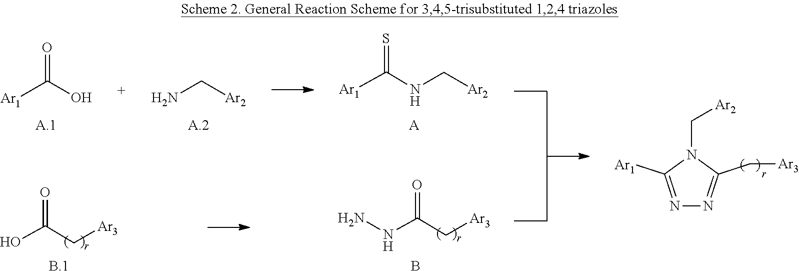

Compounds shown in Tables 1-4 were prepared following the general reaction scheme 2 above. Shown below is a more detailed reaction scheme for the preparation of Compound No. 5 (in Table 2):

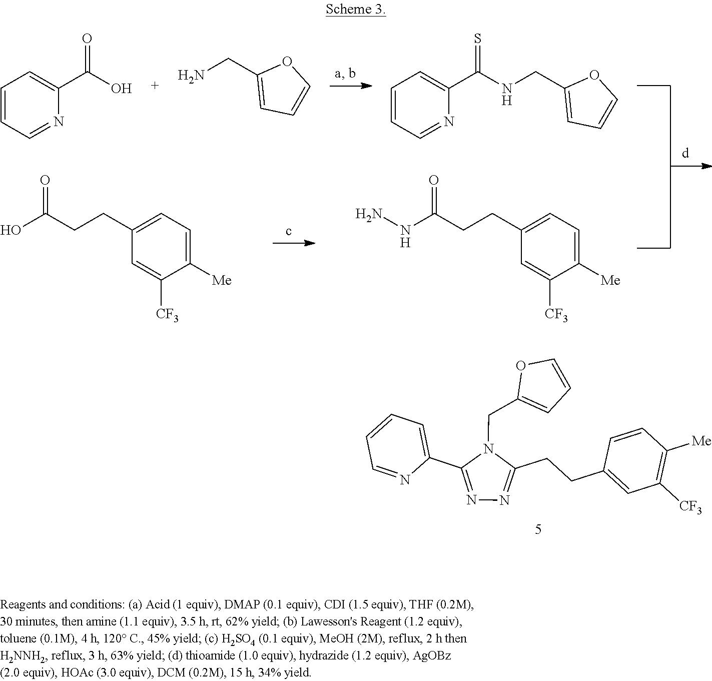

Building blocks (i.e., precursors and/or intermediate) for compounds disclosed herein without commercially available starting materials were synthesized via the Heck Reaction, the Knoevenagel Condensation, or via the Grignard Reaction. Preparation of exemplary building blocks are shown in the scheme below, where building blocks for compound 5 were prepared via Heck reaction (Scheme 4a) or Grignard Reaction (Scheme 4b), whereas the building block for Compound 34 were prepared using a Knoevenagel Condensation (Scheme 4c).

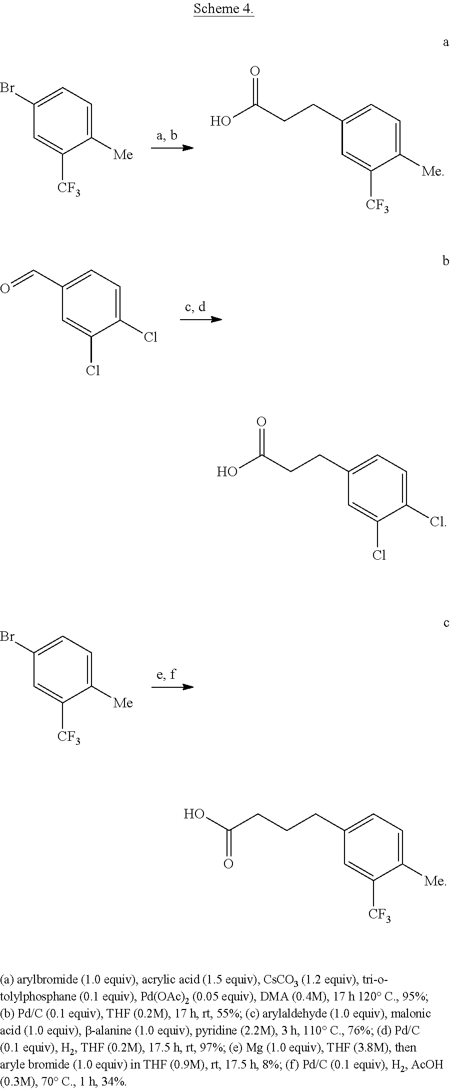 [SMILES: Cc1ccc(Br)cc1C(F)(F)F ; Cc1ccc(CCC(=O)O)cc1C(F)(F)F ; O=Cc1ccc(Cl)c(Cl)c1 ; O=C(O)CCc1ccc(Cl)c(Cl)c1 ; Cc1ccc(Br)cc1C(F)(F)F ; Cc1ccc(CCCC(=O)O)cc1C(F)(F)F]

Compounds in Table 5 and 6, which included varying linkers, were prepared via alternate methods. For example, Compound 41 in Table 5 was prepared according to the reaction scheme shown below:

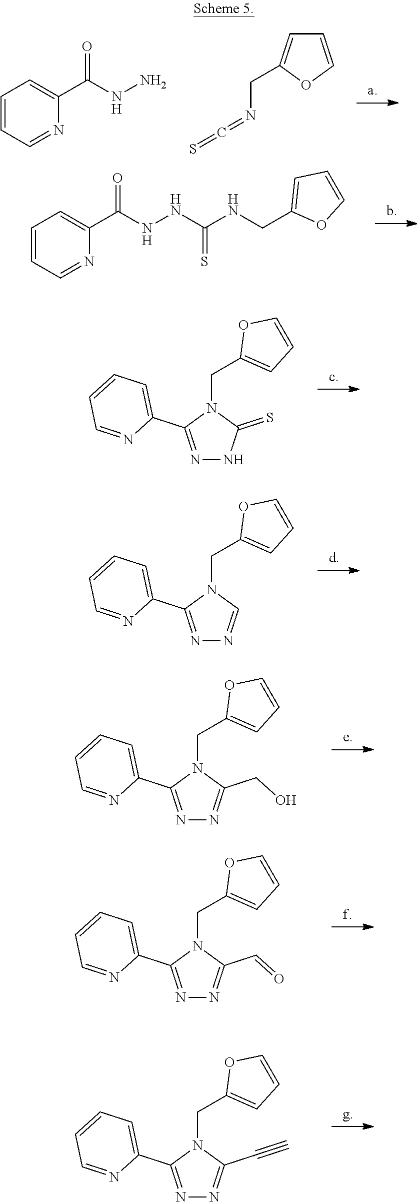

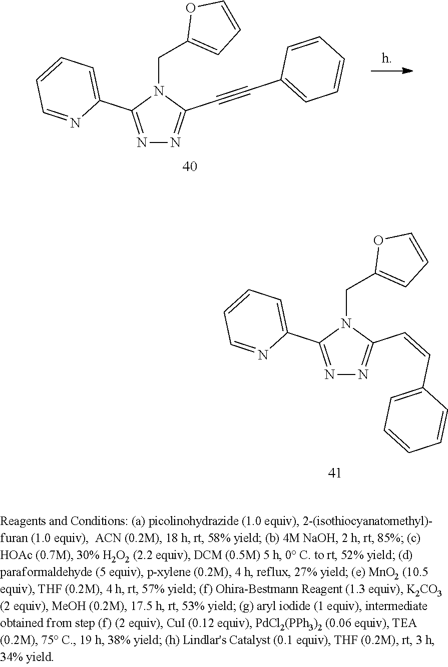 [SMILES: C(#Cc1nnc(-c2ccccn2)n1Cc1ccco1)c1ccccc1 ; C(=C/c1nnc(-c2ccccn2)n1Cc1ccco1)/c1ccccc1]

Compound 41 was synthesized via functionalizing the 3,4-disubstituted triazole as described previously.^{21,37} Once functionalized with an aldehyde, an Ohira-Bestmann alkylation followed by a Sonogashira coupling afford Compound 40. Subsequent reduction of the alkyne functionality in Compound 40 using Lindlar's catalyst afforded the cis-alkene analogue (Compound 41). This reaction sequence can be applied to all compounds in Table 5 containing a cis-alkene in linker (L), as would be apparent to a person skilled in the art.

Synthesis of compounds containing an alpha-oxygenated or alpha-fluorinated-containing linker in Table 5 were prepared as shown in the reaction scheme below.

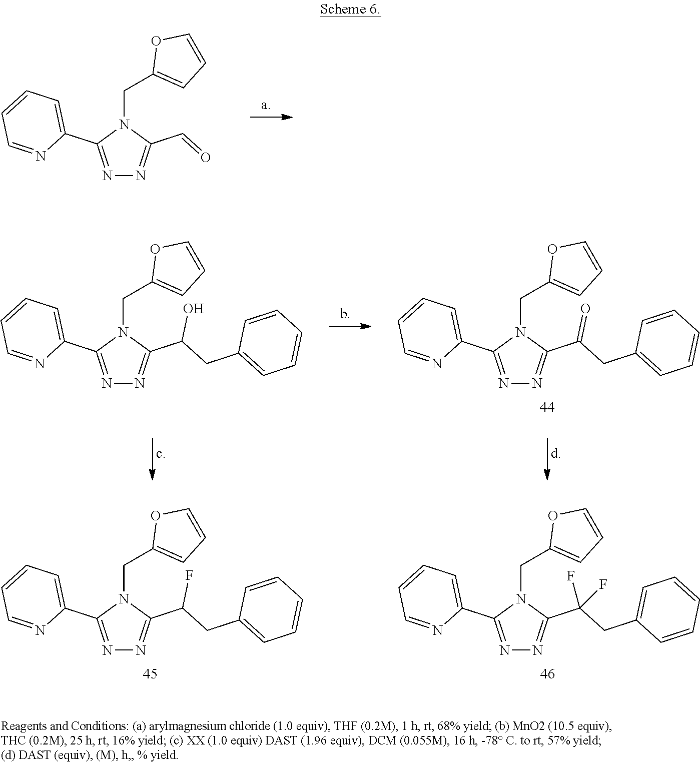 [SMILES: O=Cc1nnc(-c2ccccn2)n1Cc1ccco1 ; OC(Cc1ccccc1)c1nnc(-c2ccccn2)n1Cc1ccco1 ; O=C(Cc1ccccc1)c1nnc(-c2ccccn2)n1Cc1ccco1 ; FC(Cc1ccccc1)c1nnc(-c2ccccn2)n1Cc1ccco1 ; FC(F)(Cc1ccccc1)c1nnc(-c2ccccn2)n1Cc1ccco1]

Specifically, linkers were synthesized via addition of a Grignard Reagent into the functionalized aldehyde starting material. The resulting alcohol was then either oxidized to the ketone to obtain Compound 44 or mono-fluorinated via DAST to afford Compound 45. Alternatively, Compound 44 can be treated with DAST to obtain difluorinated Compound 46.

Synthesis of the deconstructed analogs in Table 7 that are devoid of the triazole were generally prepared via a series of amide coupling reactions. For example, the synthesis of Compound 55 is shown in the scheme below:

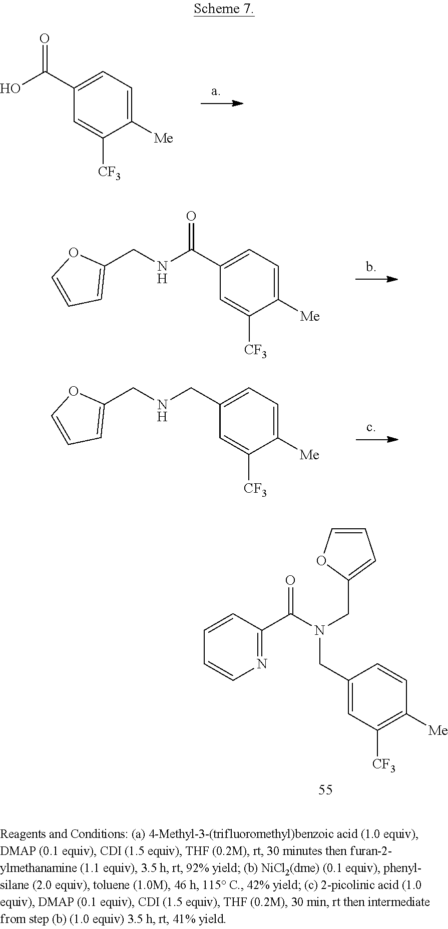

### D. Pharmaceutical Compositions

Another aspect of the disclosure is directed to a pharmaceutical composition comprising a compound as disclosed herein or a pharmaceutically acceptable salt thereof, and one or more pharmaceutically acceptable carrier(s).

In certain embodiments, compounds or salts of Formulae (A), (I), (II), and/or (III) disclosed herein, are combined with one or more additional agents to form pharmaceutical compositions. In some embodiments, compounds of Formulae (A), (I), (II), and/or (III) are already in the form of a salt. Pharmaceutical compositions may be formulated in a conventional manner using one or more physiologically acceptable carriers including excipients and auxiliaries which facilitate processing of the active compounds into preparations which can be used pharmaceutically. Proper formulation is dependent upon the route of administration chosen. Additional details about suitable excipients for pharmaceutical compositions described herein may be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

A pharmaceutical composition, as used herein, refers to a mixture of a compound or salt of Formulae (A), (I), (II), and/or (III) with any suitable substituents and functional groups disclosed herein, with other chemical components, such as carriers, stabilizers, diluents, dispersing agents, suspending agents, thickening agents, and/or excipients. The pharmaceutical composition facilitates administration of the compound to an organism. In practicing the methods of treatment or use provided herein, therapeutically effective amounts of compounds described herein are administered in a pharmaceutical composition to a mammal having a disease, disorder, or condition to be treated. In some embodiments, the mammal is a human. A therapeutically effective amount can vary widely depending on the severity of the disease, the age and relative health of the subject, the potency of the compound used and other factors. The compounds or salts of Formulae (A), (I), (II), and/or (III) with any suitable substituents and functional groups disclosed herein, can be used singly or in combination with one or more therapeutic agents as components of mixtures (as in combination therapy).

The pharmaceutical formulations described herein can be administered to a subject by multiple administration routes, including but not limited to, oral, parenteral (e.g., intravenous, subcutaneous, intramuscular), intranasal, buccal, topical, rectal, or transdermal administration routes. Moreover, the pharmaceutical compositions described herein, which include a compound of Formulae (A), (I), (II) and/or (III) with any suitable substituents and functional groups disclosed herein, can be formulated into any suitable dosage form, including but not limited to, aqueous oral dispersions, liquids, gels, syrups, elixirs, slurries, suspensions, aerosols, fast-melt formulations, effervescent formulations, lyophilized formulations, tablets, powders, pills, dragees, and capsules.

One may administer the compounds and/or compositions in a local rather than systemic manner, for example, via injection of the compound directly into an organ or tissue, often in a depot preparation or sustained release formulation. Such long acting formulations may be administered by implantation (for example, subcutaneously or intramuscularly) or by intramuscular injection. Furthermore, one may administer the drug in a targeted drug delivery system, for example, in a liposome coated with organ-specific antibody. The liposomes will be targeted to and taken up selectively by the organ. In addition, the drug may be provided in the form of a rapid release formulation, in the form of an extended release formulation or in the form of an intermediate release formulation.

Pharmaceutical compositions including a compound described herein may be manufactured in a conventional manner, such as, by way of example only, by means of conventional mixing, dissolving, granulating, dragee-making, levigating, emulsifying, encapsulating, entrapping or compression processes.

The pharmaceutical compositions will include at least one compound of Formula (I) disclosed herein, as an active ingredient in free-acid or free-base form, or in a pharmaceutically acceptable salt form.

In some embodiments, compositions provided herein may also include one or more preservatives to inhibit microbial activity. Suitable preservatives include quaternary ammonium compounds such as benzalkonium chloride, cetyltrimethylammonium bromide and cetylpyridinium chloride.

In some embodiments, the pharmaceutical solid dosage forms described herein can include a compound of Formulae (A), (I), (II) and/or (III) and one or more pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof. In still other aspects, using standard coating procedures, such as those described in Remington's Pharmaceutical Sciences, 20^{th} Edition (2000), a film coating is provided around the formulation of the compound described herein. In one embodiment, some or all of the particles of the compound described herein are coated. In another embodiment, some or all of the particles of the compound described herein are microencapsulated. In still another embodiment, the particles of the compound described herein are not microencapsulated and are uncoated.

Suitable carriers for use in the solid dosage forms described herein include, but are not limited to, acacia, gelatin, colloidal silicon dioxide, calcium glycerophosphate, calcium lactate, maltodextrin, glycerine, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, hydroxypropylmethylcellulose, hydroxypropylmethylcellulose acetate stearate, sucrose, microcrystalline cellulose, lactose, mannitol and the like.

Suitable filling agents for use in the solid dosage forms described herein include, but are not limited to, lactose, calcium carbonate, calcium phosphate, dibasic calcium phosphate, calcium sulfate, microcrystalline cellulose, cellulose powder, dextrose, dextrates, dextran, starches, pregelatinized starch, hydroxypropylmethycellulose (HPMC), hydroxypropylmethycellulose phthalate, hydroxypropylmethylcellulose acetate stearate (HPMCAS), sucrose, xylitol, lactitol, mannitol, sorbitol, sodium chloride, polyethylene glycol and the like.

Suitable disintegrants for use in the solid dosage forms described herein include, but are not limited to, natural starch such as corn starch or potato starch, a pregelatinized starch such as National 1551 or Amijel®, or sodium starch glycolate such as Promogel® or Explotab®, a cellulose such as a wood product, methylcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch and the like.

Suitable binders in the solid dosage forms described herein include, but are not limited to, carboxymethylcellulose, methylcellulose (e.g., Methocel®), hydroxypropylmethylcellulose (e.g., Hypromellose USP Pharmacoat-603), hydroxypropylmethylcellulose acetate stearate (Agoate HS LF and HS), hydroxyethylcellulose, hydroxypropylcellulose (e.g., Klucel®), ethylcellulose (e.g., Ethocel®), and microcrystalline cellulose (e.g., Avicel®), microcrystalline dextrose, amylose, magnesium aluminum silicate, polysaccharide acids, bentonites, gelatin, polyvinylpyrrolidone/vinyl acetate copolymer, crospovidone, povidone, starch, pregelatinized starch, tragacanth, dextrin, a sugar, such as sucrose (e.g., Dipac®), glucose, dextrose, molasses, mannitol, sorbitol, xylitol (e.g., Xylitab®), lactose, a natural or synthetic gum such as acacia, tragacanth, ghatti gum, mucilage of isapol husks, starch, polyvinylpyrrolidone (e.g., Povidone® CL, Kollidon® CL, Polyplasdone® XL-10 and Povidone® K-12), larch arabinogalactan, Veegum®, polyethylene glycol, waxes, sodium alginate and the like.

Suitable lubricants or glidants for use in the solid dosage forms described herein include, but are not limited to, stearic acid, calcium hydroxide, talc, corn starch, sodium stearyl fumerate, alkali-metal and alkaline earth metal salts, such as aluminum, calcium, magnesium, zinc, stearic acid, sodium stearates, magnesium stearate, zinc stearate, waxes, Stearowet®, boric acid, sodium benzoate, sodium acetate, sodium chloride, leucine, a polyethylene glycol or a methoxypolyethylene glycol such as Carbowax™, PEG 4000, PEG 5000, PEG 6000, propylene glycol, sodium oleate, glyceryl behenate, glyceryl palmitostearate, glyceryl benzoate, magnesium or sodium lauryl sulfate, and the like.

Suitable diluents for use in the solid dosage forms described herein include, but are not limited to, sugars (including lactose, sucrose and dextrose), polysaccharides (including dextrates and maltodextrin), polyols (including mannitol, xylitol and sorbitol), cyclodextrins and the like.

Suitable wetting agents for use in the solid dosage forms described herein include, for example, oleic acid, glyceryl monostearate, sorbitan monooleate, sorbitan monolaurate, triethanolamine oleate, polyoxyethylene sorbitan monooleate, polyoxyethylene sorbitan monolaurate, quaternary ammonium compounds (e.g., Polyquat 10®), sodium oleate, sodium lauryl sulfate, magnesium stearate, sodium docusate, triacetin, vitamin E TPGS and the like.

Suitable surfactants for use in the solid dosage forms described herein include, for example, sodium lauryl sulfate, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF) and the like.

Suitable suspending agents for use in the solid dosage forms described here include, but are not limited to, polyvinylpyrrolidone, e.g., polyvinylpyrrolidone K12, polyvinylpyrrolidone K17, polyvinylpyrrolidone K25, or polyvinylpyrrolidone K30, polyethylene glycol, e.g., the polyethylene glycol can have a molecular weight of about 300 to about 6000, or about 3350 to about 4000, or about 5400 to about 7000, vinyl pyrrolidone/vinyl acetate copolymer (S630), sodium carboxymethylcellulose, methylcellulose, hydroxypropylmethylcellulose, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Suitable antioxidants for use in the solid dosage forms described herein include, for example, e.g., butylated hydroxytoluene (BHT), sodium ascorbate and tocopherol.

There is considerable overlap between additives used in the solid dosage forms described herein. Thus, the above-listed additives should be taken as merely exemplary, and not limiting, of the types of additives that can be included in solid dosage forms of the pharmaceutical compositions described herein.

Liquid formulation dosage forms for oral administration can be aqueous suspensions selected from the group including, but not limited to, pharmaceutically acceptable aqueous oral dispersions, emulsions, solutions, elixirs, gels and syrups. See, e.g., Singh et al., Encyclopedia of Pharmaceutical Technology, 2^{nd} Ed., pp. 754-757 (2002).

The pharmaceutical compositions described herein may include sweetening agents such as, but not limited to, acacia syrup, acesulfame K, alitame, anise, apple, aspartame, banana, Bavarian cream, berry, black currant, butterscotch, calcium citrate, camphor, caramel, cherry, cherry cream, chocolate, cinnamon, bubble gum, citrus, citrus punch, citrus cream, cotton candy, cocoa, cola, cool cherry, cool citrus, cyclamate, cylamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, monoammonium glyrrhizinate (MagnaSweet®), maltitol, mannitol, maple, marshmallow, menthol, mint cream, mixed berry, neohesperidin DC, neotame, orange, pear, peach, peppermint, peppermint cream, Prosweet® Powder, raspberry, root beer, rum, saccharin, safrole, sorbitol, spearmint, spearmint cream, strawberry, strawberry cream, stevia, sucralose, sucrose, sodium saccharin, saccharin, aspartame, acesulfame potassium, mannitol, talin, sucralose, sorbitol, Swiss cream, tagatose, tangerine, thaumatin, tutti fruitti, vanilla, walnut, watermelon, wild cherry, wintergreen, xylitol, or any combination of these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof.

Potential excipients for intranasal formulations include formulations solutions in saline, employing benzyl alcohol or other suitable preservatives, fluorocarbons, and/or other solubilizing or dispersing agents. See, for example, Ansel, H. C. et al., Pharmaceutical Dosage Forms and Drug Delivery Systems, Sixth Ed. (1995). Preferably, these compositions and formulations are prepared with suitable nontoxic pharmaceutically acceptable ingredients. The choice of suitable carriers is highly dependent upon the exact nature of the nasal dosage form desired, e.g., solutions, suspensions, ointments, or gels. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents may also be present. Preferably, the nasal dosage form should be isotonic with nasal secretions.

For administration by inhalation, the compounds described herein may be in a form as an aerosol, a mist or a powder. Pharmaceutical compositions described herein are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of, such as, by way of example only, gelatin for use in an inhaler or insufflator may be formulated containing a powder mix of the compound described herein and a suitable powder base such as lactose or starch.

Buccal formulations that include compounds described herein may be administered using a variety of formulations which include, but are not limited to, U.S. Pat. Nos. 4,229,447; 4,596,795; 4,755,386; and 5,739,136. In addition, the buccal dosage forms described herein can further include a bioerodible (hydrolysable) polymeric carrier that also serves to adhere the dosage form to the buccal mucosa. The buccal dosage form is fabricated so as to erode gradually over a predetermined time period, wherein the delivery of the compound is provided essentially throughout. Buccal drug delivery avoids the disadvantages encountered with oral drug administration, e.g., slow absorption, degradation of the active agent by fluids present in the gastrointestinal tract and/or first-pass inactivation in the liver. With regard to the bioerodible (hydrolysable) polymeric carrier, virtually any such carrier can be used, so long as the desired drug release profile is not compromised, and the carrier is compatible with the compounds described herein, and any other components that may be present in the buccal dosage unit. Generally, the polymeric carrier comprises hydrophilic (water-soluble and water-swellable) polymers that adhere to the wet surface of the buccal mucosa. Examples of polymeric carriers useful herein include acrylic acid polymers and co, e.g., those known as “carbomers” (Carbopol®, which may be obtained from B.F. Goodrich, is one such polymer). Other components that may also be incorporated into the buccal dosage forms described herein include, but are not limited to, disintegrants, diluents, binders, lubricants, flavoring, colorants, preservatives and the like. For buccal or sublingual administration, the compositions may take the form of tablets, lozenges, or gels formulated in a conventional manner.

Transdermal formulations described herein may incorporate certain pharmaceutically acceptable excipients which are conventional in the art. In some embodiments, formulations suitable for transdermal administration of compounds described herein may employ transdermal delivery devices and transdermal delivery patches and can be lipophilic emulsions or buffered, aqueous solutions, dissolved and/or dispersed in a polymer or an adhesive.

Formulations suitable for intramuscular, subcutaneous, or intravenous injection may include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (propylene glycol, polyethylene-glycol, glycerol, Cremophor, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants. Formulations suitable for subcutaneous injection may also contain additives such as preserving, wetting, emulsifying, and dispensing agents. Prevention of the growth of microorganisms can be ensured by various antibacterial and antifungal agents, such as parabens, chlorobutanol, phenol, sorbic acid, and the like. It may also be desirable to include isotonic agents, such as sugars, sodium chloride, and the like. Prolonged absorption of the injectable pharmaceutical form can be brought about by the use of agents delaying absorption, such as aluminum monostearate and gelatin.

For intravenous injections, compounds described herein may be formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally recognized in the field. For other parenteral injections, appropriate formulations may include aqueous or nonaqueous solutions, preferably with physiologically compatible buffers or excipients. Such excipients are generally recognized in the field.

Parenteral injections may involve bolus injection or continuous infusion. Formulations for injection may be presented in unit dosage form, e.g., in ampoules or in multi-dose containers, with an added preservative. The pharmaceutical composition described herein may be in a form suitable for parenteral injection as sterile suspensions, solutions or emulsions in oily or aqueous vehicles, and may contain formulatory agents such as suspending, stabilizing and/or dispersing agents. Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions. Alternatively, the active ingredient may be in powder form for constitution with a suitable vehicle, e.g., sterile pyrogen-free water, before use.

In certain embodiments, delivery systems for pharmaceutical compounds may be employed, such as, for example, liposomes and emulsions. In certain embodiments, compositions provided herein also include an mucoadhesive polymer, selected from among, for example, carboxymethylcellulose, carbomer (acrylic acid polymer), poly(methylmethacrylate), polyacrylamide, polycarbophil, acrylic acid/butyl acrylate copolymer, sodium alginate and dextran.

In some embodiments, the compounds described herein may be administered topically and are formulated into a variety of topically administrable compositions, such as solutions, suspensions, lotions, gels, pastes, medicated sticks, balms, creams or ointments. Such pharmaceutical compounds can contain solubilizers, stabilizers, tonicity enhancing agents, buffers and preservatives.

The compounds described herein may also be formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as, but not limited to, a mixture of fatty acid glycerides, optionally in combination with cocoa butter is first melted.

In some embodiments, the compounds of Formulae (I), (II), and/or (III) disclosed herein are combined with other therapeutic agents, such as other cardiovascular agents, anti-nausea agents (or anti-emetics), anti-pruritic agents, pain relievers (including chronic and acute pain relievers and hyperalgesia), anti-inflammatory agents, neuroprotective agents, neuroleptic agents, anesthetic agents, hypertensive agents (including agents for hypoxic pulmonary hypertension), multiple sclerosis agents, muscle relaxants, and combinations thereof.

In another embodiment, the compounds of Formulae (A), (I), (II), and/or (III) disclosed herein are combined with another therapeutic agent, which are described in more detail further below.

Generally, an agent, such as a compound of Formulae (A), (I), (II), and/or (III)disclosed herein, is administered in an amount effective for treating the disease or disorder (i.e., a therapeutically effective amount). Thus, a therapeutically effective amount can be an amount that is capable of at least partially treating, preventing or reversing a disease or disorder. The dose required to obtain an effective amount may vary depending on the agent, formulation, disease or disorder, and individual to whom the agent is administered.

Determination of effective amounts may also involve in vitro assays in which varying doses of agent are administered to cells in culture and the concentration of agent effective for ameliorating some or all symptoms is determined in order to calculate the concentration required in vivo. Effective amounts may also be based in in vivo animal studies.

An agent, such as a compound of Formulae (A), (I), (II), or (III), can be administered prior to, concurrently with, and subsequent to the appearance of symptoms of a disease or disorder. In some embodiments, an agent is administered to a subject with a family history of the disease or disorder or who has a phenotype that may indicate a predisposition to a disease or disorder or who has a genotype which predisposes the subject to the disease or disorder.

In some embodiments, the compositions described herein are provided as pharmaceutical and/or therapeutic compositions. The pharmaceutical and/or therapeutic compositions of the present disclosure can be administered in a number of ways depending upon whether local or systemic treatment is desired and upon the area to be treated. Administration can be topical (including ophthalmic and to mucous membranes including vaginal and rectal delivery), pulmonary (e.g., by inhalation or insufflation of powders or aerosols, including by nebulizer, as well as: intratracheal, intranasal, epidermal and transdermal), oral or parenteral. Parenteral administration includes intravenous, intra-arterial, subcutaneous, intraperitoneal or intramuscular injection or infusion; or intracranial, e.g., intrathecal or intraventricular, administration. Compositions and formulations for topical administration can include: transdermal patches, ointments, lotions, creams, gels, drops, suppositories, sprays, liquids and powders. Conventional carriers; aqueous, powder, or oily bases; thickeners; and the like can be necessary or desirable. Compositions and formulations for oral administration include: powders or granules, suspensions or solutions in water or non-aqueous media, capsules, sachets or tablets. Thickeners, flavoring agents, diluents, emulsifiers, dispersing aids or binders can be desirable. Compositions and formulations for parenteral, intrathecal or intraventricular administration can include sterile aqueous solutions that can also contain buffers, diluents and other suitable additives such as, but not limited to, penetration enhancers, carrier compounds and other pharmaceutically acceptable carriers or excipients. Pharmaceutical and/or therapeutic compositions of the present disclosure include, but are not limited to, solutions, emulsions, and liposome containing formulations. These compositions can be generated from a variety of components that include, but are not limited to, preformed liquids, self-emulsifying solids and self-emulsifying semisolids.

The pharmaceutical and/or therapeutic formulations, which can conveniently be presented in unit dosage form, can be prepared according to conventional techniques well known in the pharmaceutical/nutraceutical industries. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product. The compositions of the present disclosure can be formulated into any of many possible dosage forms such as, but not limited to, tablets, capsules, liquid syrups, soft gels, suppositories, and enemas. The compositions of the present disclosure can also be formulated as suspensions in aqueous, non-aqueous, oil-based, or mixed media. Suspensions can further contain substances that increase the viscosity of the suspension including, for example, sodium carboxymethylcellulose, sorbitol and/or dextran. The suspension can also contain stabilizers. In one embodiment of the present disclosure, the pharmaceutical compositions can be formulated and used as foams. Pharmaceutical foams include formulations such as, but not limited to, emulsions, microemulsions, creams, jellies and liposomes. While basically similar in nature these formulations vary in the components and the consistency of the final product.

The pharmaceutical composition described herein may be in unit dosage forms suitable for single administration of precise dosages. In unit dosage form, the formulation is divided into unit doses containing appropriate quantities of one or more compound. The unit dosage may be in the form of a package containing discrete quantities of the formulation. Non-limiting examples are packaged tablets or capsules, and powders in vials or ampoules. Aqueous suspension compositions can be packaged in single-dose non-reclosable containers. Alternatively, multiple-dose reclosable containers can be used, in which case it is typical to include a preservative in the composition. By way of example only, formulations for parenteral injection may be presented in unit dosage form, which include, but are not limited to ampoules, or in multi-dose containers, with an added preservative.

Dosing and administration regimes are tailored by the clinician, or others skilled in the pharmacological arts, based upon well-known pharmacological and therapeutic considerations including, but not limited to, the desired level of therapeutic effect and the practical level of therapeutic effect obtainable. Generally, it is advisable to follow well-known pharmacological principles for administrating chemotherapeutic agents (e.g., it is generally advisable to not change dosages by more than 50% at time and no more than every 3-4 agent half-lives). For compositions that have relatively little or no dose-related toxicity considerations, and where maximum efficacy is desired, doses in excess of the average required dose are not uncommon. This approach to dosing is commonly referred to as the “maximal dose” strategy. In certain embodiments, the compounds are administered to a subject at a dose of about 0.01 mg/kg to about 200 mg/kg, more preferably at about 0.1 mg/kg to about 100 mg/kg, even more preferably at about 0.5 mg/kg to about 50 mg/kg. When the compounds described herein are co-administered with another agent (e.g., as sensitizing agents), the effective amount may be less than when the agent is used alone. Dosing may be once per day or multiple times per day for one or more consecutive days.

### E. Methods of Use

The disclosure provides compounds and methods for treating a subject suffering from a disease and/or condition that has been identified as being associated with the kappa opioid receptor. In one aspect, the present disclosure provides a method for treating a disease or condition mediated by the kappa opioid receptor, the method comprising administering a therapeutically effective amount of a compound provided herein or a pharmaceutical composition described herein to a subject in need thereof. In some embodiments, the disease or condition to be treated is selected from the group consisting of pain, itching, addiction, depression, myocardial infarction, pruritus, inflammation, edema, nausea, neuro-inflammation, stroke, hypoxic pulmonary hypertension, ocular inflammation, glaucoma, multiple sclerosis, osteoarthritis, rheumatoid arthritis, inflammatory bowel disease and eczema. In certain embodiments, the subject does not experience any dysphoria or sedation.

In some embodiments, the disclosure provides a method of treating such a disease and/or condition comprising administering a therapeutically effective amount of a compound or salt described herein, for example, a compound or salt of Formulae (A), (I), (II), and/or (III) disclosed herein, to a subject in need thereof. In some embodiments, the compound or salt being administered is a kappa opioid agonist as disclosed herein. In some embodiments, the compound or salt being administered is a g-protein biased kappa opioid agonist as disclosed herein, for example, a compound or salt of Formulae (A), (I), (II), and/or (III).

In some embodiments, such diseases and/or conditions can be treated by activating or inactivating signaling of the kappa opioid receptor. In some embodiments, such diseases and/or disorders can be treated by agonizing or inactivating signaling of the kappa opioid receptor.

The kappa opioid receptor-associated disease or conditions treatable with the compounds disclosed herein can be any kappa opioid receptor-associated condition. Such diseases and conditions to be treated include, but are not limited to, acute and chronic pain, inflammation, cardiovascular disease (e.g., congestive heart failure, stroke, hypertension), liver cirrhosis, edema, ileus, tussis, eye diseases (i.e., glaucoma, ocular inflammation), itching, addiction, depression, myocardial infarction, pruritus, nausea, neuro-inflammation, hypoxic pulmonary hypertension, multiple sclerosis, osteoarthritis, rheumatoid arthritis, inflammatory bowel disease, substance abuse disorder, and eczema.

For instance, the kappa opioid receptor-associated pain can be neuropathic pain, somatic pain, visceral pain or cutaneous pain. Some diseases or conditions are associated with more than one form of pain, e.g., postoperative pain can have any or all of neuropathic, somatic, visceral, and cutaneous pain components, depending upon the type and extent of surgical procedure employed.

The kappa opioid receptor-associated inflammation can be any inflammatory disease or condition including, but not limited to sinusitis, rheumatoid arthritis tenosynovitis, bursitis, tendonitis, lateral epicondylitis, adhesive capsulitis, osteomyelitis, osteoarthritic inflammation, inflammatory bowel disease (IBD), irritable bowel syndrome (IBS), ocular inflammation, otitic inflammation or autoimmune inflammation.

The kappa opioid receptor-associated pruritus can be any pruritic disease or condition such as, for instance, ocular pruritus, e.g., associated with conjunctivitis, otitic pruritus, pruritus associated with end-stage renal disease, where many patients are receiving kidney dialysis, and other forms of cholestasis, including primary biliary cirrhosis, intrahepatic cholestasis of pregnancy, chronic cholestatic liver disease, uremia, malignant cholestasis, jaundice, as well as dermatological conditions such as eczema (dermatitis), including atopic or contact dermatitis, psoriasis, polycythemia vera, lichen planus, lichen simplex chronicus, pediculosis (lice), thyrotoxicosis, tinea pedis, urticaria, scabies, vaginitis, anal pruritis associated with hemorrhoids and, as well as insect bite pruritis and drug-induced pruritis, such as mu opioid-induced pruritis.

The kappa opioid receptor-associated edema can be any edematous disease or condition such as, for instance, edema due to congestive heart disease or to a syndrome of inappropriate antidiuretic hormone (ADH) secretion.

Kappa opioid receptor-associated ileus can be any ileus disease or condition including, but not limited to, post-operative ileus and opioid-induced bowel dysfunction.

Kappa opioid receptor-associated neuropathic pain can be any neuropathic pain, such as, for instance, trigeminal neuralgia, diabetic pain, viral pain such as herpes zoster-associated pain, chemotherapy-induced pain, nerve-encroaching metastatic cancer pain, neuropathic pain associated with traumatic injury and surgical procedures, as well as variants of headache pain that are thought to have a neuropathic component, e.g., migraine.

Kappa opioid-associated pain also includes ocular pain, such as that following photo-refractive keratectomy (PRK), ocular laceration, orbital floor fracture, chemical burns, corneal abrasion or irritation, or pain associated with conjunctivitis, corneal ulcers, scleritis, episcleritis, sclerokeratitis, herpes zoster ophthalmicus, interstitisal keratitis, acute iritis, keratoconjunctivitis sicca, orbital cellulites, orbital pseudotumor, pemphigus, trachoma or uveitis.

Kappa opioid-associated pain also includes throat pain, particularly associated with inflammatory conditions, such as allergic rhinitis, acute bronchitis, the common cold, contact ulcers, herpes simplex viral lesions, infectious mononucleosis, influenza, laryngeal cancer, acute laryngitis, acute necrotizing ulcerative gingivitis, peritonsillar abscess, pharyngeal burns, pharyngitis, reflux laryngopharyngitis, acute sinusitis, and tonsillitis.

Kappa opioid receptor-associated pain can be arthritic pain, kidney-stone, urinary tract stone, gallstone, and bile duct stone pain, dysmenorrhea, uterine cramping, endometriosis, mastitis, dyspepsia, post-surgical pain (such as, for instance, from appendectomy, open colorectal surgery, hernia repair, prostatectomy, colonic resection, gastrectomy, splenectomy, colectomy, colostomy, pelvic laparoscopy, tubal ligation, hysterectomy, vasectomy or cholecystectomy), post medical procedure pain (such as, for instance, after colonoscopy, cystoscopy, hysteroscopy or cervical or endometrial biopsy), otitic pain, breakthrough cancer pain, and pain associated with a GI disorder such as IBD or IBS or other inflammatory conditions, particularly of the viscera (e.g., gastro-esophageal reflux disease, pancreatitis, acute pyelonephritis, ulcerative colitis, acute pyelo-nephritis, cholecystitis, cirrhosis, hepatic abscess, hepatitis, duodenal or gastric ulcer, esophagitis, gastritis, gastroenteritis, colitis, diverticulitis, intestinal obstruction, ovarian cyst, pelvic inflammatory disease, perforated ulcer, peritonitis, prostatitis, interstitial cystitis), or exposure to toxic agents, such as insect toxins, or inflammation due to the effects of drugs such as salicylates or NSAIDs.

Kappa opioid receptor-associated pain also includes hyperalgesia, which is believed to be caused by changes in the milieu of the peripheral sensory terminal occur secondary to local tissue damage. Tissue damage (e.g., abrasions, burns) and inflammation can produce significant increases in the excitability of polymodal nociceptors (C fibers) and high threshold mechanoreceptors (Handwerker et al. (1991) Proceeding of the VIth World Congress on Pain, Bond et al., eds., Elsevier Science Publishers BV, pp. 59-70; Schaible et al. (1993) Pain 55:5-54). This increased excitability and the exaggerated responses of sensory afferents is believed to underlie hyperalgesia, where the pain response is the result of an exaggerated response to a stimulus. The importance of the hyperalgesic state in the post-injury pain state has been repeatedly demonstrated and appears to account for a major proportion of the post-injury/inflammatory pain state. See for example, Woold et al. (1993) Anesthesia and Analgesia 77:362-79; Dubner et al. (1994) In, Textbook of Pain, Melzack et al., eds., Churchill-Livingstone, London, pp. 225-242.

In another embodiment the kappa opioid receptor-associated condition is pain, inflammation (such as rheumatoid arthritic inflammation, osteoarthritic inflammation, IBD inflammation, IBS inflammation, ocular inflammation, otitic inflammation or autoimmune inflammation), pruritus (such as atopic dermatitis, kidney-dialysis-associated pruritus, ocular pruritus, otitic pruritus, insect bite pruritus, or opioid-induced pruritus), edema, ileus, tussis or glaucoma. In one aspect, the pain is a neuropathic pain (such as trigeminal neuralgia, migraine, diabetic pain, viral pain, chemotherapy-induced pain or metastatic cancer pain), a somatic pain, a visceral pain or a cutaneous pain. In another aspect the pain is arthritic pain, kidney-stone pain, uterine cramping, dysmenorrhea, endometriosis, dyspepsia, post-surgical pain, post medical procedure pain, ocular pain, otitic pain, breakthrough cancer pain or pain associated with a GI disorder, such as IBD or IBS. In another aspect the pain is pain associated with surgery, wherein the surgery is pelvic laparoscopy, tubal ligation, hysterectomy and cholecystecomy. Alternatively, the pain can be pain associated with a medical procedure, such as for instance, colonoscopy, cystoscopy, hysteroscopy or endometrial biopsy. In a specific aspect, the atopic dermatitis can be psoriasis, eczema or contact dermatitis. In another specific aspect, the ileus is post-operative ileus or opioid-induced bowel dysfunction.

The disclosure also provides a method of treating or preventing a neuro-degenerative disease or condition in a subject, wherein the method includes administering to the subject a composition that includes an effective amount of a composition as disclosed herein. The neurodegenerative disease or condition can be any neurodegenerative disease or condition, such as for instance, ischemia, anoxia, stroke, brain injury, spinal cord injury or reperfusion injury. Alternatively, the neurodegenerative disease or condition can be a neurodegenerative disease of the eye. Particular neurodegenerative diseases of the eye treatable or preventable by the method of the invention include glaucoma, macular degeneration, retinal ischemic disease and diabetic neuropathy.

In some embodiments, the compound disclosed herein is a neurological modulator (e.g., anti-nociceptive agent, antidepressant, anxiolytic, antipruritic).

In some embodiments, the disclosure provides methods of prevention or treatment of certain neuronal diseases and conditions, such as diseases and conditions having a neurodegenerative component. Compounds as disclosed herein can be administered in an amount effective to protect neuronal cells against the effects of pathology or injury that would lead to neurodegeneration and/or neuronal cell death of the untreated cells. For example, several diseases or conditions of the eye that have a neurodegenerative component can be prevented or treated by administration of an effective amount of compounds as disclosed herein. Such diseases and conditions of the eye include glaucoma, macular degeneration, retinal ischemic disease and diabetic neuropathy. Progression of these diseases and conditions is believed to involve neurodegeneration or neuronal cell death, for example by programmed cell death (apoptosis) in which the neuronal cells are committed to a pathway that without intervention would lead to cell death. It has been found that development or progression of these diseases and conditions can be prevented, or at least slowed, by treatment with kappa opioid receptor agonists. This improved outcome is believed to be due to neuroprotection by the kappa opioid receptor agonists. See for instance, Kaushik et al. “Neuroprotection in Glaucoma” (2003) J. Postgraduate Medicine vol. 49 (1): pp. 90-95.

Moreover, the compounds as disclosed herein can be administered by methods disclosed herein for the treatment or prevention of any hyperalgesic condition associated with burns, abrasions, bruises, abrasions (such as corneal abrasions), contusions, frostbite, rashes, acne, insect bites/stings, skin ulcers (for instance, diabetic ulcers or a decubitus ulcers), mucositis, inflammation, gingivitis, bronchitis, laryngitis, sore throat, shingles, fungal irritation (such as athlete's foot or jock itch), fever blisters, boils, Plantar's warts or vaginal lesions (such as vaginal lesions associated with mycosis or sexually transmitted diseases).

Hyperalgesic conditions associated with post-surgery recovery can also be addressed by administration of the composition disclosed herein. The hyperalgesic conditions associated with post-surgery recovery can be any hyperalgesic conditions associated with post-surgery recovery, such as for instance, radial keratectomy, tooth extraction, lumpectomy, episiotomy, laparoscopy and arthroscopy.

Hyperalgesic conditions associated with inflammation are also addressable by administration of the compounds disclosed herein. Periodontal inflammation, orthodontic inflammation, inflammatory conjunctivitis, hemorrhoids and venereal inflammations can be treated or prevented by topical or local administration of the compounds disclosed herein.

In some embodiments, the methods described above further comprise co-administration of a second therapeutic agent. Such a therapeutic agent can be administered at the same time as the compound disclosed herein or can be administered before or after administration of the compound disclosed herein. A skilled artisan would be aware that the mode and timing of administration and the dose being administered can vary depending on the therapeutic agent being administered.

Examples of therapeutic agents being co-administered include, but are not limited to, other opioids, cannabinoids, antidepressants, anticonvulsants, neuroleptics, antihistamines, acetaminophen, corticosteroids, ion channel blocking agents, non-steroidal anti-inflammatory drugs (NSAIDs), and diuretics.

Suitable opioids, include, without limitation, alfentanil, alphaprodine, anileridine, bremazocine, buprenorphine, butorphanol, codeine, conorphone, dextromoramide, dextropropoxyphene, dezocine, diamorphine, dihydrocodeine, dihydromorphine, diphenoxylate, dipipanone, doxpicomine, ethoheptazine, ethylketazocine, ethylmorphine, etorphine, fentanyl, hydrocodone, hydromorphone, ketobemidone, levomethadyl, levorphanol, lofentanil, loperamide, meperidine (pethidine), meptazinol, methadone, morphine, morphine-6-glucuronide, nalbuphine, nalorphine, nicomorphine, oxycodone, oxymorphone, pentazocine, phenazocine, phenoperidine, piritramide, propiram, propoxyphene, remifentanil, sufentanil, tilidate, tonazocine, and tramadol.

Additional opioids include opioids with substantial agonist activity at the mu opioid receptor, such as morphine, fentanyl, hydromorphone, or oxycodone, together with the compounds disclosed herein, for the purpose of a mu opioid dose-sparing effect, where the dose of the mu opioid is reduced to minimize common mu opioid side effects, particularly in opioid-naïve patients. Such side effects include constipation, nausea, vomiting, sedation, respiratory depression, pruritus (itching), mental confusion, disorientation and cognitive impairment, urinary retention, biliary spasm, delirium, myoclonic jerks, and seizures. The selection of the reduced mu opioid dose requires expert clinical judgment, and depends on the characteristics of the various mu opioids, as well as patient characteristics such as pain intensity, patient age, coexisting disease, current drug regimen and potential drug interactions, prior treatment outcomes, and patient preference (McCaffery, M. and Pasero, C., Pain Clinical Manual, Second Edition, Mosby, 1999).

Cannabinoids suitable for administration with or incorporation into the pharmaceutical compositions of the invention, include any natural cannabinoid, such as for instance, tetrahydrocannabinol (THC), or a THC derivative, or a synthetic cannabinoid, such as, for instance, levonantradol, marinol, nabilone, rimonabant or sativex.

Suitable antidepressants that can be co-administered with or incorporated into the pharmaceutical compositions of the invention, include for example, tricyclic antidepressants such as imipramine, desipramine, trimipramine, protriptyline, nortriptyline, amitriptyline, doxepin, and clomipramine; atypical antidepressants such as amoxapine, maprotiline, trazodone, bupropion, and venlafaxine; serotonin-specific reuptake inhibitors such as fluoxetine, sertraline, paroxetine, citalopram and fluvoxamine; norepinephrine-specific reuptake inhibitors such as reboxetine; or dual-action antidepressants such as nefazodone and mirtazapine.

Suitable neuroleptics that can be co-administered with or incorporated into the pharmaceutical compositions of the invention, include any neuroleptic, for example, a compound with D2 dopamine receptor antagonist activity such as domperidone, metoclopramide, levosulpiride, sulpiride, thiethylperazine, ziprasidone, zotepine, clozapine, chlorpromazine, acetophenazine, carphenazine, chlorprothixene, fluphenazine, loxapine, mesoridazine, molindone, prochlorperazine, pimozide, piperacetazine, perchlorperazine, thioridazine, thiothixene, trifluoperazine, triflupromazine, pipamperone, amperozide, quietiapine, melperone, remoxipride, haloperidol, rispiridone, olanzepine, sertindole, ziprasidone, amisulpride, prochlorperazine, and thiothixene.

Anticonvulsants such as phenobarbital, phenytoin, primidone, carbamazepine, ethosuximide, lamotrigine, valproic acid, vigabatrin, felbamate, gabapentin, levetiracetam, oxcarbazepine, remacemide, tiagabine, and topiramate can also usefully be incorporated into the pharmaceutical compositions of the invention.

Muscle relaxants such as methocarbamol, orphenadrine, carisoprodol, meprobamate, chlorphenesin carbamate, diazepam, chlordiazepoxide and chlorzoxazone; anti-migraine agents such as sumitriptan, analeptics such as caffeine, methylphenidate, amphetamine and modafinil; antihistamines such as chlorpheniramine, cyproheptadine, promethazine and pyrilamine, as well as corticosteroids such as methylprednisolone, betamethasone, hydrocortisone, prednisolone, cortisone, dexamethasone, prednisone, alclometasone, clobetasol, clocortolone, desonide, desoximetasone, diflorasone, fluocinolone, fluocinonide, flurandrenolide, fluticasone, flouromethalone, halcinonide, halobetasol, loteprednol, mometasone, prednicarbate, and triamcinolone can also be incorporated into the pharmaceutical compositions of the invention.

Ion channel blocking agents such as, for instance, the sodium ion channel blocker, carbamazepine, as commonly used in the treatment of tinnitus, arrhythmia, ischemic stroke and epilepsy can be co-administered with or incorporated into the pharmaceutical compositions of the invention. Alternatively, or in addition, calcium ion channel blockers, such as ziconotide, can also be used, as can antagonists of the ion channel associated with the NMDA receptor, such as ketamine. There is evidence that at least some of these ion channel blockers can potentiate the analgesic effects of the kappa agonist and thereby reduce the dose required for affective pain relief. See for instance, Wang et al., 2000, Pain 84: 271-81.

Suitable NSAIDs, or other non-opioid compounds with anti-inflammatory and/or analgesic activity, that can be co-administered with or incorporated into the pharmaceutical compositions of the invention include, but are not limited to one or more of the following: aminoarylcarboxylic acid derivatives such as etofenamate, meclofenamic acid, mefanamic acid, niflumic acid; arylacetic acid derivatives such as acemetacin, amfenac, cinmetacin, clopirac, diclofenac, fenclofenac, fenclorac, fenclozic acid, fentiazac, glucametacin, isoxepac, lonazolac, metiazinic acid, naproxin, oxametacine, proglumetacin, sulindac, tiaramide and tolmetin; arylbutyric acid derivatives such as butibufen and fenbufen; arylcarboxylic acids such as clidanac, ketorolac and tinoridine. arylpropionic acid derivatives such as bucloxic acid, carprofen, fenoprofen, flunoxaprofen, ibuprofen, ibuproxam, oxaprozin, phenylalkanoic acid derivatives such as flurbiprofen, piketoprofen, pirprofen, pranoprofen, protizinic acid and tiaprofenic acid; pyranocarboxylic acids such as etodolac; pyrazoles such as mepirizole; pyrazolones such as clofezone, feprazone, mofebutazone, oxyphinbutazone, phenylbutazone, phenyl pyrazolidininones, suxibuzone and thiazolinobutazone; salicylic acid derivatives such as aspirin, bromosaligenin, diflusinal, fendosal, glycol salicylate, mesalamine, 1-naphthyl salicylate, magnesium salicylate, olsalazine and salicylamide, salsalate, and sulfasalazine; thiazinecarboxamides such as droxicam, isoxicam and piroxicam others such as F-acetamidocaproic acid, acetaminophen, s-adenosylmethionine, 3-amino-4-hydroxybutyric acid, amixetrine, bendazac, bucolome, carbazones, cromolyn, difenpiramide, ditazol, hydroxychloroquine, indomethacin, ketoprofen and its active metabolite 6-methoxy-2-naphthylacetic acid; guaiazulene, heterocylic aminoalkyl esters of mycophenolic acid and derivatives, nabumetone, nimesulide, orgotein, oxaceprol, oxazole derivatives, paranyline, pifoxime, 2-substituted-4, 6-di-tertiary-butyl-s-hydroxy-1,3-pyrimidines, proquazone and tenidap, and cox-2 (cyclooxygenase II) inhibitors, such as celecoxib or rofecoxib.

Suitable diuretics that can be co-administered with or incorporated into the pharmaceutical preparations of the invention, include, for example, inhibitors of carbonic anhydrase, such as acetazolamide, dichlorphenamide, and methazolamide; osmotic diuretics, such as glycerin, isosorbide, mannitol, and urea; inhibitors of Na+-K+-2Cl− symport (loop diuretics or high-ceiling diuretics), such as furosemide, bumetanide, ethacrynic acid, torsemide, axosemide, piretanide, and tripamide; inhibitors of Na+-Cl− symport (thiazide and thiazidelike diuretics), such as bendroflumethiazide, chlorothiazide, hydrochlorothiazide, hydroflumethazide, methyclothiazide, polythiazide, trichlormethiazide, chlorthalidone, indapamide, metolazone, and quinethazone; and, in addition, inhibitors of renal epithelial Na+ channels, such as amiloride and triamterene, and antagonists of mineralocorticoid receptors (aldosterone antagonists), such as spironolactone, canrenone, potassium canrenoate, and eplerenone, which, together, are also classified as K+-sparing diuretics

In some embodiments, any of the above therapeutic agents can also be formulated with the composition disclosed herein to obtain pharmaceutical compositions as described above.

In accordance with the purpose(s) of the currently disclosed subject matter, as embodied and broadly described herein, one aspect relates to a method of preventing or treating a disease or condition mediated by the kappa opioid receptor, the method comprising administering a therapeutically effective amount of a compound of Formula (A) to a subject in need thereof, wherein the compound of Formula (A) is:

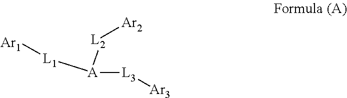

or a pharmaceutically acceptable salt thereof, wherein:
A is selected from the group consisting of

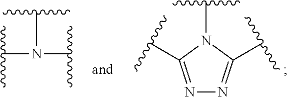

Ar₁ is selected from the group consisting of

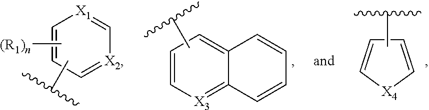

wherein X₁, X₂ and X₃ are each independently selected from the group consisting of —N— and —CH—, and X₄ is selected from the group consisting of —CH₂—, —NH—, —O—, —S—, and —N(CH₃)—, wherein R₁, in each instance, is independently selected from the group consisting of —Cl, —Br, —F, —CF₃, —OH, —CN, —NO₂, —NH₂, and (C₁-C₆)alkyl, and n is 1, 2 or 3;
Ar₂ is selected from the group consisting of

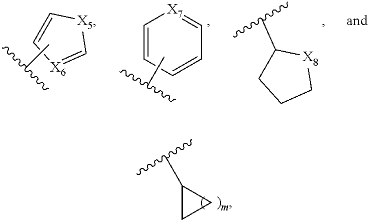

wherein X₅ and X₈ are each independently is selected from the group consisting of —CH₂—, —NH—, —O—, and —S—, and X₆ and X₇ are each independently selected from the group consisting of —N— and —CH—, and wherein m is 1, 2, 3, 4, 5, or 6;
Ar₃ is selected from the group consisting of

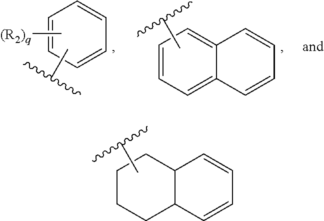

wherein R₂, in each instance, is independently selected from the group consisting of —Cl, —Br, —F, —CF₃, —OH, —CN, —NO₂, —NH₂, —O(C₁-C₆)alkyl, and —(C₁-C₆)alkyl, and q is 1, 2, 3, 4, or 5; and L₁, L₂ and L₃ are each independently selected from the group consisting of a bond, —C(═O)—, —(CH₂)ᵣ—, —C≡C—, —CH═CH—, —C(R_{4b})(R₄ₐ)CH₂—, —CH₂C(R_{4b})(R₄ₐ)—, —C(═O)CH₂—, —CH₂CH(R₅)—, and —CH(Rₓ)—, wherein r is 1, 2, 3, 4, 5, or 6, and R₄ₐ, R_{4b}, R₅, and Rₓ are each independently selected from the group consisting of —H, —Cl, —Br, —F, —CF₃, —OH, —CN, —NO₂, —NH₂, and —(C₁-C₆)alkyl, or R₄ₐ and R_{4b} are taken together with their intervening atom(s) to form C₃-C₇ carbocyclyl.

In accordance with the purpose(s) of the currently disclosed subject matter, as embodied and broadly described herein, one aspect relates to a method of preventing or treating a disease or condition mediated by the kappa opioid receptor, the method comprising administering a therapeutically effective amount of a compound of Formula (I) to a subject in need thereof, wherein the compound of Formula (I) is:

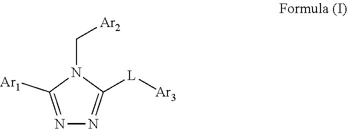

or a pharmaceutically acceptable salt thereof, wherein
Ar₁ is selected from the group consisting of

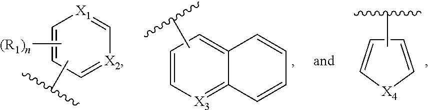

wherein X₁, X₂, and X₃ are each independently selected from the group consisting of —N— and —CH—, and X₄ is selected from the group consisting of —CH₂—, —NH—, —O—, —S—, and —N(CH₃)—, wherein R₁, in each instance, is independently selected from the group consisting of, —Cl, —Br, —F, —CF₃, —OH, —CN, —NO₂, —NH₂, and —(C₁-C₆)alkyl, and n is 1, 2 or 3;
Ar₂ is selected from the group consisting of

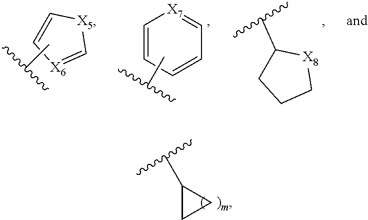

wherein X₅ and X₈ are each independently selected from the group consisting of —CH₂—NH—, —O—, and —S—, and X₆ and X₇ are each independently selected from the group consisting of —N— and —CH—, and wherein m is 1, 2, 3, 4, 5, or 6;
Ar₃ is selected from the group consisting of

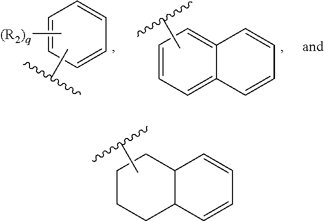

wherein R₂, in each instance, is selected from the group consisting of —Cl, —Br, —F, —CF₃, —OH, —CN, —NO₂, —NH₂, —O(C₁-C₆)alkyl, and —(C₁-C₆)alkyl, and q is 1, 2, 3, 4, or 5; and
L is selected from the group consisting of a bond, —(CH₂)ᵣ—, —C≡C—, —CH═CH—, —C(R_{4b})(R₄ₐ)CH₂—, —CH₂C(R_{4b})(R₄ₐ)—, —C(═O)CH₂—, and —CH₂CH(R₅)—, wherein r is 1, 2, 3, 4, 5, or 6, and R₄ₐ, R_{4b} and R₅ are independently selected from the group consisting of —H, —Cl, —Br, —F, —CF₃, —OH, —CN, —NO₂, —NH₂, and —(C₁-C₆)alkyl, or R₄ₐ and R_{4b} are taken together with their intervening atom(s) to form C₃-C₇ carbocyclyl.

### EXAMPLES

### Example 1: Assays Used for Structure-Activity Relationship Studies in Tables 1-8

Compounds in Tables 1-8 were examined for G-protein activation using the GTPγS biochemical assay.²⁹ In additions, compounds in Tables 1-7 were examined for β-arrestin recruitment using the PathHunter® DiscoverX β-arrestin recruitment assay.³⁰ Bias was determined by fitting to the operational model as previously described (Zhou et al., 2013, 22). Microsomal stability was determined using mouse hepatic microsomes and LC/MS detections over time (Zhou et al., 2013, 22).

### Example 2: Compounds Tested in a Standard Puritis Mouse Model

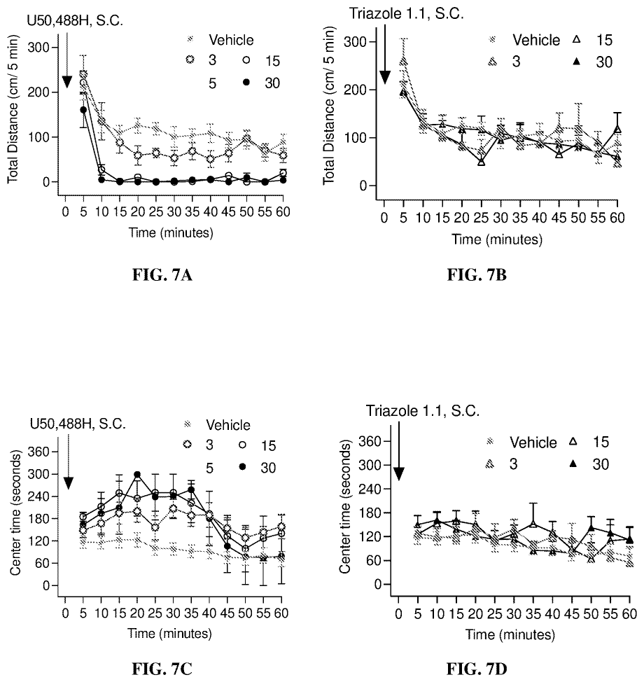

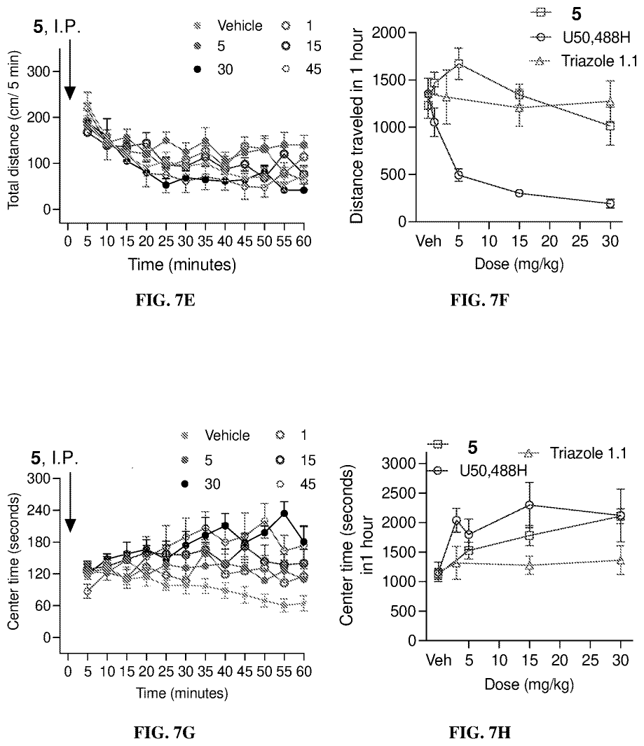

Compounds 5, U50,488H, and Triazole 1.1 were tested for their anti-puritis activity in a standard puritis mouse model (adult male C57BL6/J mice from Jackson Labs were used in all assays). This involves injection of compound 15 min prior to injection of the pruritis-inducing agent: chloroquine phosphate. The number of scratches are recorded by two investigators blinded to treatment. The data shown in FIGS. 6, 7A, and 7B shows that 5 decreases scratching following an injection of chloroquine phosphate (40 mg/kg, s.c. nape of neck). In addition, 5 exhibits essentially equipotent anti-puritic activity as the conventional agonist: U50,488H and KOR agonist Triazole 1.1. This was described in Brust et al., 2016.(23)

### Example 3: Compounds Tested in a Standard Mouse Model for Sedation (Open Field Test Box Model)

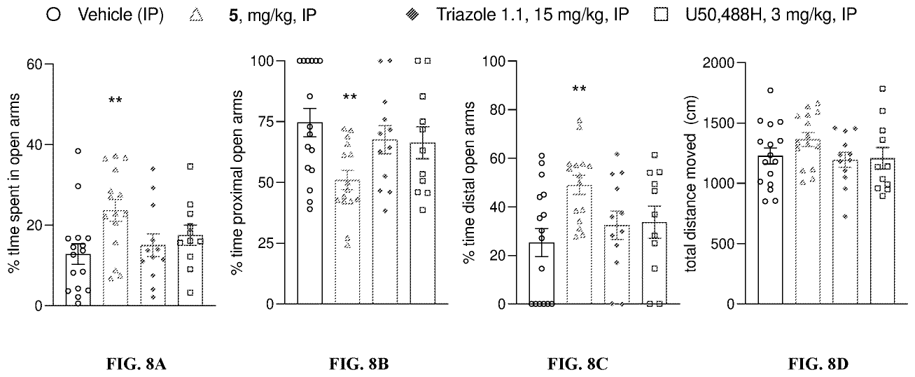

Compounds 5, U50,488H, and Triazole 1.1 were tested for their ability to induce sedation in a standard mouse model for determining sedation: the open field test activity monitor. Adult male C57BL6/J mice are given drug and immediately placed into the box which monitors beam breaks in real time to assess movement. The data shown in FIG. 8 shows that unlike U50,488H Triazole 1.1 does not induce sedation. Compound 5 also does not induce sedation. On the bottom four panels, it can be seen that U50,488H increases the time spent in the center of the open field box, whereas Triazole 1.1 did not effect center time. However, animals with 5 also had increased center time. An increase in time spent in the center, without sedation, is a very desirable property of a KOR agonist. This was described in Brust et al., 2016.(23)

### Example 4: Compounds Tested in a Standard Mouse Model for Anxiety (Elevated Plus Maze Mouse Model)

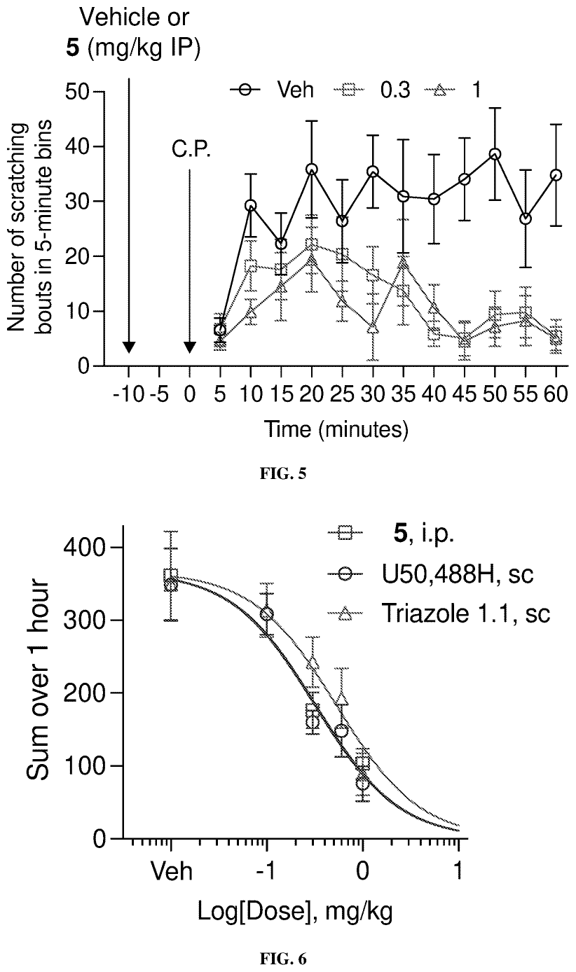

Compounds 5, U50,488H, and Triazole 1.1 were tested for their ability to exhibit anxiolytic properties in a standard mouse model of anxiety: the elevated plus maze mouse model. Adult male C57BL6 mice were injected 30 minutes prior to the start of the test and then assessed by automated video analysis for the amount of time spent in open (2) vs closed (2) arms. Drug dosing was randomized such that vehicle was present in each cohort of 4 testing days and the person running the test was blinded to drug dosing. The data in FIG. 5 shows that at the doses tested, none of the agonists showed sedation during the test (5 min), 30 min after drug treatment. However, only 5 increased the time spent in the distal ends of the open arms in this test. These data suggest that 5 has anxiolytic properties. **one-way ordinary anova; p<0.01.

### REFERENCES

(1) Hedegaard, H. Drug Overdose Deaths in the United States, 1999-2020. NCHS Data Brief 2021.
(2) Matthes, H. W. D.; Maldonado, R.; Simonin, F.; Valverde, O.; Slowe, S.; Kitchen, I.; Befort, K.; Dierich, A.; Le Meur, M.; Dollé, P.; et al. Loss of morphine-induced analgesia, reward effect and withdrawal symptoms in mice lacking the p-opioid-receptor gene. Nature 1996, 383 (6603), 819-823. DOI: 10.1038/383819a0.
(3) Sora, I.; Takahashi, N.; Funada, M.; Ujike, H.; Revay, R. S.; Donovan, D. M.; Miner, L. L.; Uhl, G. R. Opiate receptor knockout mice define μ receptor roles in endogenous nociceptive responses and morphine-induced analgesia. Proceedings of the National Academy of Sciences 1997, 94 (4), 1544-1549. DOI: 10.1073/pnas.94.4.1544 (accessed 2022/12/13).
(4) Kivell, B.; Prisinzano, T. E. Kappa opioids and the modulation of pain. Psychopharmacology 2010, 210 (2), 109-119. DOI: 10.1007/s00213-010-1819-6.
(5) Kardon, Adam P.; Polgár, E.; Hachisuka, J.; Snyder, Lindsey M.; Cameron, D.; Savage, S.; Cai, X.; Karnup, S.; Fan, Christopher R.; Hemenway, Gregory M.; et al. Dynorphin Acts as a Neuromodulator to Inhibit Itch in the Dorsal Horn of the Spinal Cord. Neuron 2014, 82 (3), 573-586. DOI: 10.1016/j.neuron.2014.02.046 (accessed 2022/12/13).
(6) Inan, S.; Cowan, A. Kappa opioid agonists suppress chloroquine-induced scratching in mice. European Journal of Pharmacology 2004, 502 (3), 233-237. DOI: https://doi.org/10.1016/j.ejphar.2004.09.010.
(7) Glick, S. D.; Maisonneuve, I. M.; Raucci, J.; Sydney, A. Kappa opioid inhibition of morphine and cocaine self-administration in rats. Brain Research 1995, 681 (1), 147-152. DOI: https://doi.org/10.1016/0006-8993(95)00306-B.
(8) Mague, S. D.; Pliakas, A. M.; Todtenkopf, M. S.; Tomasiewicz, H. C.; Zhang, Y.; Stevens, W. C.; Jones, R. M.; Portoghese, P. S.; Carlezon, W. A. Antidepressant-Like Effects of κ-Opioid Receptor Antagonists in the Forced Swim Test in Rats. Journal of Pharmacology and Experimental Therapeutics 2003, 305 (1), 323. DOI: 10.1124/jpet.102.046433.
(9) Chadzinska, M.; Hermsen, T.; Savelkoul, H. F. J.; Verburg-van Kemenade, B. M. L. Cloning of opioid receptors in common carp (Cyprinus carpio L.) and their involvement in regulation of stress and immune response. Brain, Behavior, and Immunity 2009, 23 (2), 257-266. DOI: https://doi.org/10.1016/j.bbi.2008.10.003.
(10) Pfeiffer, A.; Brantl, V.; Herz, A.; Emrich, H. M. Psychotomimesis Mediated by κ Opiate Receptors. Science 1986, 233 (4765), 774-776. DOI: 10.1126/science.3016896 (accessed 2022/12/14).
(11) Land, B. B.; Bruchas Mr Fau—Lemos, J. C.; Lemos Jc Fau—Xu, M.; Xu M Fau—Melief, E. J.; Melief Ej Fau—Chavkin, C.; Chavkin, C. The dysphoric component of stress is encoded by activation of the dynorphin kappa-opioid system. The Journal of Neuroscience 2008, 28 (2), 407-414. From 2008 Jan. 9.
(12) Kenakin, T.; Christopoulos, A. Signalling bias in new drug discovery: detection, quantification and therapeutic impact. Nature Reviews Drug Discovery 2013, 12 (3), 205-216. DOI: 10.1038/nrd3954.
(13) Che, T.; Dwivedi-Agnihotri, H.; Shukla, A. K.; Roth, B. L. Biased ligands at opioid receptors: Current status and future directions. Science Signaling 2021, 14 (677), eaav0320. DOI: 10.1126/scisignal.aav0320 (accessed 2022/12/14).
(14) Lahti, R. A.; Mickelson, M. M.; McCall, J. M.; Von Voigtlander, P. F. [3H]U-69593 a highly selective ligand for the opioid x receptor. European Journal of Pharmacology 1985, 109 (2), 281-284. DOI: https://doi.org/10.1016/0014-2999(85)90431-5.
(15) Pasternak, G. W. Multiple opiate receptors: [3H]ethylketocyclazocine receptor binding and ketocyclazocine analgesia. Proceedings of the National Academy of Sciences 1980, 77 (6), 3691-3694. DOI: 10.1073/pnas.77.6.3691 (accessed 2022/12/14).
(16) Rives, M.-L.; Rossillo, M.; Liu-Chen, L.-Y.; Javitch, J. A. 6′-Guanidinonaltrindole (6′-GNTI) Is a G Protein-biased k-Opioid Receptor Agonist That Inhibits Arrestin Recruitment. Journal of Biological Chemistry 2012, 287 (32), 27050-27054. DOI: 10.1074/jbc.C112.387332 (acccessed 2022/12/14).
(17) Roth, B. L.; Baner, K.; Westkeamper, R.; Siebert, D.; Rice, K. C.; Steinber, S.; Ernsberger, P.; Rothman, R. B. Salvinorin A: a potent naturally occurring nonnitrogenous kappa opioid selective agonist. PNAS 2002, 99, 11934-11939.
(18) Wu, H.; Wacker D Fau—Mileni, M.; Mileni M Fau—Katritch, V.; Katritch V Fau—Han, G. W.; Han Gw Fau—Vardy, E.; Vardy E Fau—Liu, W.; Liu W Fau—Thompson, A. A.; Thompson Aa Fau—Huang, X.-P.; Huang Xp Fau—Carroll, F. I.; Carroll Fi Fau—Mascarella, S. W.; et al. Structure of the human κ-opioid receptor in complex with JDTic. Nature 2012, (1476-4687 (Electronic)). From 2012 Mar. 21.
(19) Che, T.; Majumdar, S.; Zaidi, S. A.; Ondachi, P.; McCorvy, J. D.; Wang, S.; Mosier, P. D.; Uprety, R.; Vardy, E.; Krumm, B. E.; et al. Structure of the Nanobody-Stabilized Active State of the Kappa Opioid Receptor. Cell 2018, (1097-4172 (Electronic)). From 2018 Jan. 11.
(20) Wang, Y.; Zhuang, Y.; DiBerto, J. F.; Zhou, X. E.; Schmitz, G. P.; Yuan, Q.; Jain, M. K.; Liu, W.; Melcher, K.; Jiang, Y.; et al. Structures of the entire human opioid receptor family. Cell 2023, (186), 413-427. DOI: 10.1016/j.cell.2022.12.026 (acccessed 2023/01/19).
(21) Frankowski, K. J.; Hedrick, M. P.; Gosalia, P.; Li, K.; Shi, S.; Whipple, D.; Ghosh, P.; Prisinzano, T. E.; Schoenen, F. J.; Su, Y.; et al. Discovery of Small Molecule Kappa Opioid Receptor Agonist and Antagonist Chemotypes through a HTS and Hit Refinement Strategy. ACS Chem Neurosci 2012, 3 (3), 221-236. DOI: 10.1021/cn200128x.
(22) Zhou, L.; Lovell, K. M.; Frankowski, K. J.; Slauson, S. R.; Phillips, A. M.; Streicher, J. M.; Stahl, E.; Schmid, C. L.; Hodder, P.; Madoux, F.; et al. Development of functionally selective, small molecule agonists at kappa opioid receptors. J Biol Chem 2013, 288 (51), 36703-36716. DOI: 10.1074/jbc.M113.504381.
(23) Brust, T. B.; Morgenwekc, J.; Kim, S. A.; Rose, J. H.; Locke, J. L.; Schmid, C. L.; Zhou, L.; Stahl, E. L.; Cameron, M. D.; Scarry, S. M.; et al. Biased Agonists of the Kappa Opioid Receptor Suppress Pain and Itch Without Causing Sedation and Dysphoria. Science Signalling 2016, 9.
(24) Huskinson, S. L.; Platt, D. M.; Brasfield, M.; Follett, M. E.; Prisinzano, T. E.; Blough, B. E.; Freeman, K. B. Quantification of observable behaviors induced by typical and atypical kappa-opioid receptor agonists in male rhesus monkeys. Psychopharmacology 2020, (1432-2072 (Electronic)). From 2020 July
(25) Huskinson, S. L.; Platt, D. M.; Zamarripa, C. A.; Dunaway, K.; Brasfield, M.; Prisinzano, T. E.; Blough, B. E.; Freeman, K. B. The G-protein biased kappa opioid agonists, Triazole 1.1 and nalfurafine, produce non-uniform behavioral effects in male rhesus monkeys. Pharmacology Biochemistry and Behavior 2022, (1873-5177 (Electronic)). From 2022 June
(26) Zamarripa, C. A.; Pareek, T.; Schrock, H. M.; Prisinzano, T. E.; Blough, B. E.; Sufka, K. J.; Freeman, K. B. The kappa-opioid receptor agonist, Triazole 1.1, reduces oxycodone self-administration and enhances oxycodone-induced thermal antinociception in male rats. Psychopharmacology 2021, 238 (12), 3463-3476. DOI: 10.1007/s00213-021-05965-x.
(27) Lovell, K. M.; Frankowski, K. J.; Stahl, E. L.; Slauson, S. R.; Yoo, E.; Prisinzano, T. E.; Aube, J.; Bohn, L. M. Structure-activity relationship studies of functionally selective kappa opioid receptor agonists that modulate ERK 1/2 phosphorylation while preserving G protein over betaarrestin2 signaling bias. ACS Chem Neurosci 2015, 6 (8), 1411-1419. DOI: 10.1021/acschemneuro.5b00092.
(28) Frankowski, K. J.; Brust, T.; Lovell, K. M.; Yoo, E.; Bohn, L. M.; Aubé, J. Structure-activity relationship investigation of triazole-based kappa opioid receptor agonists. Medicinal Chemistry Research 2021, 30 (7), 1386-1396. DOI: 10.1007/s00044-021-02746-1.
(29) Harrison, C.; Traynor, J. R. The [35S]GTPgammaS binding assay: approaches and applications in pharmacology. Life Sciences 2003 (0024-3205 (Print)). From 2003 Dec. 12.
(30) Zhao, X.; Jones, A.; Olson, K. R.; Peng, K.; Wehrman, T.; Park, A.; Mallari, R.; Nebalasca, D.; Young, S. W.; Xiao, S.-H. A Homogeneous Enzyme Fragment Complementation-Based β-Arrestin Translocation Assay for High-Throughput Screening of G-Protein-Coupled Receptors. Journal of Biomolecular Screening 2008, 13 (8), 737-747. DOI: 10.1177/1087057108321531 (acccessed 2022/12/15).
(31) Khan, M. I. H.; Sawyer, B. J.; Akins, N. S.; Le, H. V. A systematic review on the kappa opioid receptor and its ligands: New directions for the treatment of pain, anxiety, depression, and drug abuse. European Journal of Medicinal Chemistry 2022, (1768-3254 (Electronic)). From 2022 Dec. 5.
(32) Purser, S.; Moore, P. R.; Swallow, S.; Gouverneur, V. Fluorine in medicinal chemistry. Chemical Society Reviews 2008, 37 (2), 320-330, 10.1039/B610213C. DOI: 10.1039/B610213C.
(33) Prisinzano, T. E.; Tidgewell K Fau—Harding, W. W.; Harding, W. W. Kappa opioids as potential treatments for stimulant dependence. The AAPS Journal 2005, (1550-7416 (Electronic)). From 2005 Oct. 19.
(34) Butelman, E. R.; Yuferov, V.; Kreek, M. J. κ-opioid receptor/dynorphin system: genetic and pharmacotherapeutic implications for addiction. Trends in Neurosciences 2012, 35 (10), 587-596. DOI: https://doi.org/10.1016/j.tins.2012.05.005.
(35) Bibian, M.; Blayo, A.-L.; Moulin, A.; Martinez, J.; Fehrentz, J.-A. Multi-gram scale mercury-free synthesis of optically pure 3,4,5-trisubstituted 1,2,4-triazoles using silver benzoate. Tetrahedron Letters 2010, 51 (19), 2660-2663. DOI: https://doi.org/10.1016/j.tetlet.2010.03.037.
(36) Ben Haj Salah, K.; Legrand, B.; Bibian, M.; Wenger, E.; Fehrentz, J.-A.; Denoyelle, S. Synthesis of [1,2,4]Triazolo[4,3-a]piperazin-6-ones: An Approach to the Triazole-Fused Ketopiperazine Scaffold. Organic Letters 2018, 20 (11), 3250-3254. DOI: 10.1021/acs.orglett.8b01112.
(37) Ivanova, N. V.; Sviridov, S. I.; Shorshnev, S. V.; Stepanov, A. E. A Convenient Synthesis of 4,5-Disubstituted 1,2,4-Triazoles Functionalized in Position 3. Synthesis 2006, 2006 (01), 156-160. DOI: 10.1055/s-2005-921754.

That which is claimed is:

1. A compound of Formula (A): 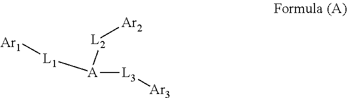
or a pharmaceutically acceptable salt thereof, wherein
A is selected from the group consisting of 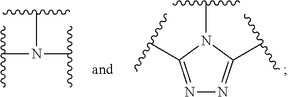
Ar₁ is selected from the group consisting of 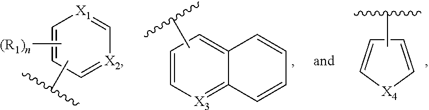
wherein X₁, X₂ and X₃ are each independently selected from the group consisting of —N— and —CH—, and X₄ is selected from the group consisting of —CH₂—, —NH—, —O—, —S—, and —N(CH₃)—, wherein R₁, in each instance, is independently selected from the group consisting of —Cl, —Br, —F, —CF₃, —OH, —CN, —NO₂, —NH₂, and (C₁-C₆)alkyl, and n is 1, 2 or 3;
Ar₂ is selected from the group consisting of 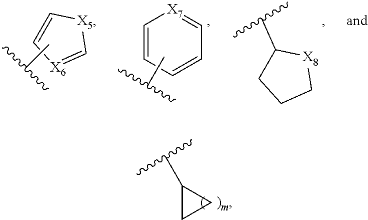
wherein X₅ and X₈ are each independently is selected from the group consisting of —CH₂—, —NH—, —O—, and —S—, and X₆ and X₇ are each independently selected from the group consisting of —N— and —CH—, and wherein m is 1, 2, 3, 4, 5, or 6;
Ar₃ is selected from the group consisting of 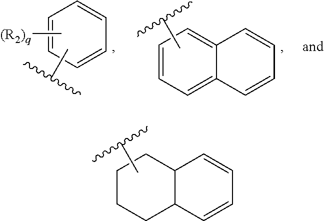
wherein R₂, in each instance, is independently selected from the group consisting of —Cl, —Br, —F, —CF₃, —OH, —CN, —NO₂, —NH₂, —O(C₁-C₆)alkyl, and —(C₁-C₆)alkyl, and q is 1, 2, 3, 4, or 5; and
L₁, L₂ and L₃ are each independently selected from the group consisting of a bond, —C(═O)—, —(CH₂)ᵣ—, —C≡C—, —CH═CH—, —C(R_{4b})(R₄ₐ)CH₂—, —CH₂C(R_{4b})(R₄ₐ)—, —C(═O)CH₂—, —CH₂CH(R₅)—, and —CH(Rₓ)—, wherein r is 1, 2, 3, 4, 5, or 6, and R₄ₐ, R_{4b}, R₅, and Rₓ are each independently selected from the group consisting of —H, —Cl, —Br, —F, —CF₃, —OH, —CN, —NO₂, —NH₂, and —(C₁-C₆)alkyl, or R₄ₐ and R_{4b} are taken together with their intervening atom(s) to form C₃-C₇ carbocyclyl.

2. The compound of claim 1, having the structure of Formula (A-1): 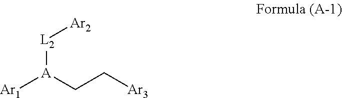
or a pharmaceutically acceptable salt thereof, wherein L₂ is a bond, —(CH₂)ᵣ—, or —CH(R^{x})—, wherein r is 1 or 2, and R^{x} is selected from the group consisting of —H and —(C₁-C₆)alkyl.

3. A compound with the structure of Formula (I): 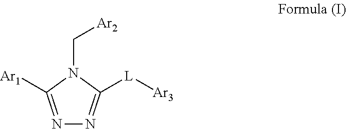
or a pharmaceutically acceptable salt thereof, wherein
Ar₁ is selected from the group consisting of 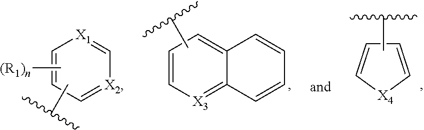
wherein X₁, X₂ and X₃ are each independently selected from the group consisting of —N— and —CH—, and X₄ is selected from the group consisting of —CH₂—, —NH—, —O—, —S—, and —N(CH₃)—, wherein R₁, in each instance, is selected from —Cl, —Br, —F, —CF₃, —OH, —CN, —NO₂, —NH₂, and —(C₁-C₆)alkyl, and n is 1, 2 or 3;
Ar₂ is selected from the group consisting of 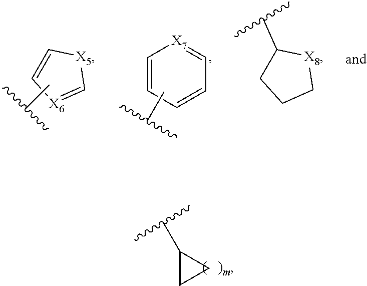
wherein X₅ and X₈ are each independently is selected from the group consisting of —CH₂—, —NH—, —O—, and —S—, and X₆ and X₇ are each independently selected from the group consisting of —N— and —CH—, and wherein m is 1, 2, 3, 4, 5, or 6;
Ar₃ is selected from the group consisting of 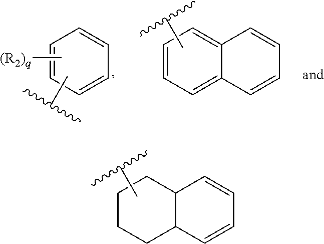
wherein R₂, in each instance, is selected from —Cl, —Br, —F, —CF₃, —OH, —CN, —NO₂, —NH₂, —O(C₁-C₆)alkyl, and —(C₁-C₆)alkyl, and q is 1, 2, 3, 4, or 5; and
L is selected from the group consisting of a bond, —(CH₂)ᵣ—, —C≡C—, —CH═CH—, —CR_{4b}(R₄ₐ)CH₂, —C(═O)CH₂—, and —CH₂CH(R₅)—, wherein r is 1, 2, 3, 4, 5, or 6, and R₄ₐ, R_{4b} and R₅ are independently selected from the group consisting of —H, —Cl, —Br, —F, —CF₃, —OH, —CN, —NO₂, —NH₂, and —(C₁-C₆)alkyl, or R₄ₐ and R_{4b} are taken together with their intervening atom(s) to form C₃-C₇ carbocyclyl.

4. The compound of claim 3, wherein L is —(CH₂)ᵣ— and r is 2.

5. The compound of any one of claims 1-4, wherein Ar₁ is

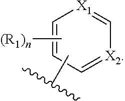

6. The compound of claim 5, wherein X₂ is —CH—.

7. The compound of claim 6, wherein R_{I}, in each instance, is —CH₃, and n is 1.

8. The compound of claim 4, wherein Ar₁ is selected from the group consisting of

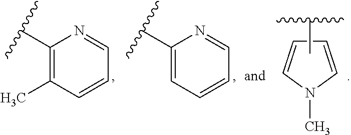

9. The compound of any one of claims 1-8, having the structure of Formula (II): 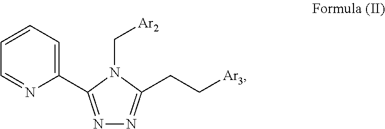
or a pharmaceutically acceptable salt thereof.

10. The compound of claim 9, wherein Ar₃ is

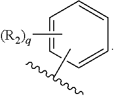

11. The compound of claim 10, wherein R₂, in each instance, is independently selected from the group consisting of —Cl, —Br, —CF₃, and —(C₁-C₆)alkyl.

12. The compound of claim 11, wherein q is 1 or 2.

13. The compound of claim 12, wherein Ar₃ is selected from the group consisting of

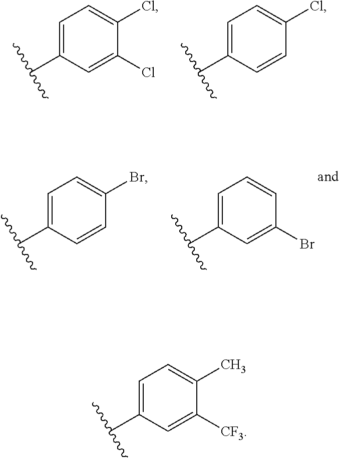 [SMILES: *c1ccc(Cl)c(Cl)c1 ; *c1ccc(Cl)cc1 ; *c1ccc(Br)cc1 ; *c1cccc(Br)c1 ; *c1ccc(C)c(C(F)(F)F)c1]

14. The compound of any one of claims 1-13, having the structure of Formula (III): 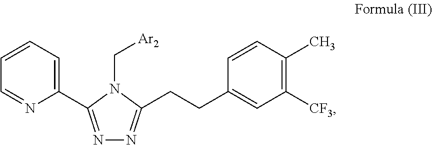
or a pharmaceutically acceptable salt thereof.

15. The compound of claim 14, wherein Ar₂ is 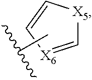
X₅ is selected from the group consisting of —NH—, —O—, and —S—, and X₆ is —CH— or —N—.

16. The compound of claim 14, wherein Ar₂ is

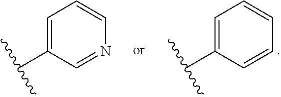 [SMILES: *c1cccnc1 ; "or" ; *c1ccccc1 ; "."]

17. The compound of claim 1 or 3, wherein Ar₂ is selected from the group consisting of

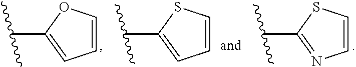

18. The compound of claim 3, wherein said compound has the structure 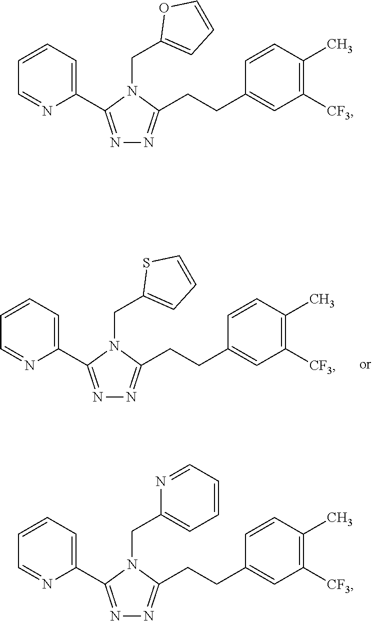 [SMILES: Cc1ccc(CCc2nnc(-c3ccccn3)n2Cc2ccco2)cc1C(F)(F)F ; Cc1ccc(CCc2nnc(-c3ccccn3)n2Cc2cccs2)cc1C(F)(F)F ; "or" ; Cc1ccc(CCc2nnc(-c3ccccn3)n2Cc2ccccn2)cc1C(F)(F)F]
or a pharmaceutically acceptable salt thereof.

19. The compound of claim 1, wherein said compound is a compound provided in any one of Tables 1-8.

20. The compound of claim 3, wherein said compound is a compound provided in any one of Tables 1-7.

21. A pharmaceutical composition comprising a compound according to any one of the preceding claims or a pharmaceutically acceptable salt thereof, and one or more pharmaceutically acceptable carrier(s).

22. A method for treating a disease or condition mediated by the kappa opioid receptor, the method comprising administering a therapeutically effective amount of a compound of any one of claims 1-20 or a pharmaceutical composition of claim 21 to a subject in need thereof.

23. The method of claim 22, wherein the disease or condition to be treated is selected from the group consisting of pain, itching, addiction, depression, myocardial infarction, pruritus, inflammation, edema, nausea, neuro-inflammation, stroke, hypoxic pulmonary hypertension, ocular inflammation, glaucoma, multiple sclerosis, osteoarthritis, rheumatoid arthritis, inflammatory bowel disease and eczema.

24. The method of claim 22 or 23, wherein the subject does not experience any dysphoria or sedation.
